(19) United States
(12) Patent Application Publication
(10) Pub. No.: US 2023/0001379 A1
(43) Pub. Date: Jan. 5, 2023

(54) POLYCRYSTALLINE METAL-ORGANIC FRAMEWORK MEMBRANES FOR SEPARATION OF MIXTURES

(71) Applicant: National University of Singapore, Singapore (SG)

(72) Inventors: Dan Zhao, Singapore (SG); Linzhi Zhai, Singapore (SG); Yahui Cai, Singapore (SG)

(21) Appl. No.: 17/763,960

(22) PCT Filed: Sep. 17, 2020
(86) PCT No.: PCT/SG2020/050532

§ 371 Date: Mar. 25, 2022

(30) Foreign Application Priority Data
Sep. 30, 2019 (SG) 10201909137Q

(51) Int. Cl. B01J 20/22; B01J 20/28; B01J 20/30; B01J 20/34; B01D 65/02; B01D 71/06; B01D 67/00; B01D 69/02; B01D 69/10; C02F 1/44; B01D 61/36; B01D 17/00

(52) U.S. Cl. CPC B01J 20/226; B01J 20/28033; B01J 20/28011; B01J 20/3085; B01J 20/3475; B01D 65/02; B01D 71/06; B01D 67/0079; B01D 69/02; B01D 69/108; C02F 1/448; B01D 61/362; B01D 17/085; B01D 2325/04; C02F 2101/34

(57) ABSTRACT

Disclosed herein is a polycrystalline metal-organic framework membrane comprising a substrate material having a surface and a polycrystalline metal-organic framework attached to the surface of the substrate material, wherein the polycrystalline metal-organic framework is formed from a secondary building unit having the formula Ia or IIb and a ligand as defined in the application.

### FIELD OF INVENTION

The invention relates to a polycrystalline metal-organic framework membrane.

### BACKGROUND

The listing or discussion of a prior-published document in this specification should not necessarily be taken as an acknowledgement that the document is part of the state of the art or is common general knowledge.

Polycrystalline metal-organic framework (MOF) membranes have attracted wide interests because of their uniform yet tunable pore size that may allow molecular sieving separation. Currently, most of the reported polycrystalline MOF membranes are for gas separation, while their applications in liquid separation especially those involving water are severely limited by their insufficient water stability. Li et al. pioneered the water-stable polycrystalline UiO-66(Zr) membrane on alumina hollow fiber for desalination, and subsequently fabricated UiO-66(Zr) membranes on pre-structured yttria-stabilized zirconia hollow fibers, with a separation factor of 55.8±1.0 in separating 10 wt. % water/ethanol feed solution. Uemiya et al. reported a separation factor of approximately 4.3 of the UiO-66(Zr) membrane in separating water/ethanol mixture.

Although UiO-66(Zr)-MOFs have high water stability, the easily formed structural defects within UiO-66(Zr) crystals pose a great challenge for the preparation of high quality polycrystalline MOF membranes. It was shown that the inherent defects of UiO-66(Zr)—(OH)₂ membranes can be improved by a post-synthetic defect healing method, displaying 74.9% increase in Na⁺ rejection compared with the pristine membrane (ACS Appl. Mater. Interfaces, 9 (2017) 37848-37855). Caro et al. healed the defects of UiO-66 membrane by coating with a polymer layer, presenting a greater H₂/CH₄ selectivity with a separation factor of 80.0. Despite these progresses, the preparation of water-stable and defect-free polycrystalline MOF membranes remains a great challenge.

MOFs composed of rare-earth (RE) cations have been attracting attention due to their high water stability and rich functionality. Recently, Eddaoudi et al. disclosed a series of RE-MOFs (i.e., Tb³⁺, Eu³⁺, and Y³⁺) with face-centered cubic (fcu) topology, in which 12-connected RE-containing secondary building units (SBUs) do not display versatile connectivity commonly observed in Zr-SBUs (i.e., 12-, 10-, 8-, or 6-connectivity). In addition, Sun et al. confirmed that the 12-connected RE-MOFs are stable due to their lower defect tolerance compared with Zr₆-MOFs. In addition, the bond energies of RE-O (i.e., Sm—, Er—, Dy—, Y—, and Gd—O are in the range of 573-715 kJ mol-1) are lower than that of Zr—O (776 kJ mol⁻¹).

Although membrane processes have been commercially established in reverse-osmosis desalination and certain gas separations, organic solvent nanofiltration (OSN, aka solvent-resistant nanofiltration, SRNF) has led to several technical challenges for conventional membranes, such as NF270, NP030, BW30, and ORAK polymeric membranes. Specifically, most of the reported polymeric membranes tend to swell, plasticize, or even dissolve in the presence of aggressive organic solvents, leading to loss of morphological structure and severely compromised separation performance. On the other hand, inorganic membranes such as zeolite membranes have excellent solvent resistance making them suitable for OSN. Nevertheless, the limited chemical tunability and relatively small pore size of zeolite membranes confine their applications mainly in dehydration.

Most reported polycrystalline MOF membranes are fabricated using rigid and expensive ceramic substrates with low packing density. There is therefore a need to find suitable substrates with high packing density and good solvent resistance to expedite the progress of polycrystalline MOF membranes for OSN.

### SUMMARY OF INVENTION

Aspects and embodiments of the invention will now be described by reference to the following numbered clauses.

1. A polycrystalline metal-organic framework membrane comprising:
a substrate material having a surface; and
a polycrystalline metal-organic framework attached to the surface of the substrate material, wherein the polycrystalline metal-organic framework is formed from:
a secondary building unit having the formula Ia or Ib:

M₆O₄(OH)₄ Ia,
where M is selected from Zr, Hf, and Ti; or

M′₆(OH)₈ Ib
where M′ is selected from Sm, Y, Dy, Er, Gd, and Ce; and
a ligand selected from fumaric acid, butynedioic acid, squaric acid, naphthalene-2,6-dicarboxylic acid, [2,2′-bipyridine]-5,5′-dicarboxylic acid, or a ligand having formula II:

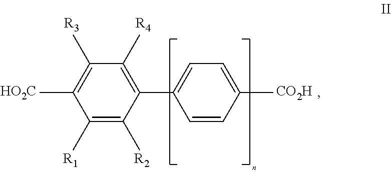

where:

R₁ is selected from H, halo, OR₅ₐ, SR_{5b}, C₁ to C₅ alkyl, NO₂, NR_{5c}R_{5d}, SO₃H, CF₃, or CO₂H;

R₂ is selected from H, halo, OR₆ₐ, SR_{6b}, C₁ to C₅ alkyl, NO₂, NR_{6c}R_{6d}, SO₃H, CF₃ or CO₂H;

R₃ is selected from H, halo, OR₇ₐ, SR_{7b}, C₁ to C₅ alkyl, NO₂, NR_{7c}R_{7d}, SO₃H, CF₃, or CO₂H;

R₄ is selected from H, halo, OR₈ₐ, SR_{8b}, C₁ to C₅ alkyl, NO₂, NR_{8c}R_{8d}, SO₃H, CF₃ or CO₂H;

R_{5a-5d}, R_{6a-6d}, R_{7a-7d}, R_{7a-7d} are each independently selected from H or C₁ to C₅ alkyl; or

R₁ and R₂ or R₃ and R₄ form, together with the carbon atoms to which they are attached to a C aromatic ring; and n is 0, 1 or 2.

2. The membrane according to Clause 1, wherein the substrate material is selected from one or more of a polymer, a ceramic (e.g. alumina), a carbon cloth, a metal, and a metal oxide.

3. The membrane according to Clause 2, wherein, when the substrate material is alumina, then the secondary building unit has formula Ia, where M is Zr or, more particularly, Hf or Ti or, yet more particularly, the secondary building unit has formula Ib.

4. The membrane according to any one of the preceding clauses, wherein the polycrystalline metal-organic framework is a UiO-66-type metal-organic framework.

5. The membrane according to any one of the preceding clauses, wherein:

R₁ is selected from H, halo, OR₅ₐ, SR_{5b}, C₁ to C₅ alkyl, NO₂, NR_{5c}R_{5d}, SO₃H, CF₃, or CO₂H;

R₂ is selected from H, OR₆ₐ, SR_{6b}, CF₃ or CO₂H;

R₃ is selected from H, halo, OR₇ₐ, SR_{7b}, C₁ to C₅ alkyl, NR_{7c}R_{7d}, SO₃H, CF₃, or CO₂H; and

R₄ is selected from H, F, OR₈ₐ, SR_{8b}, CF₃ or CO₂H.

6. The membrane according to any one of the preceding clauses, wherein:

when two or more of R₁ to R₄ are not H, then the non-H substituents are identical to each other; and/or

n is 0 or 1 (e.g. n is 0).

7. The membrane according to any one of the preceding clauses, wherein the ligand is selected from fumaric acid, butynedioic acid, squaric acid, naphthalene-2,6-dicarboxylic acid, [2,2′-bipyridine]-5,5′-dicarboxylic acid, terephthalic acid, 2-fluoroterephthalic acid, 2-chloroterephthalic acid, 2-bromoterephthalic acid, 2-iodoterephthalic acid, 2-hydroxyterephthalic acid, 2-mercaptoterephthalic acid, 2-methylterephthalic acid, 2-nitroterephthalic acid, 2-aminoterephthalic acid, 2-sulfoterephthalic acid, 2-(trifluoromethyl)terephthalic acid, benzene-1,2,4-tricarboxylic acid, 2,3-dihydroxyterephthalic acid, 2,3-dimercaptoterephthalic acid, 2,5-difluoroterephthalic acid, 2,5-dichloroterephthalic acid, 2,5-dibromoterephthalic acid, 2,5-diiodoterephthalic acid, 2,5-terephthalic acid, 2,5-dihydroxyterephthalic acid, 2,5-dimercaptoterephthalic acid, 2,5-dimethylterephthalic acid, 2,5-diaminoterephthalic acid, 2,5-bis(trifluoromethyl)terephthalic acid, 2,5-diemthoxyterephthalic acid, benzene-1,2,4,5-tetracarboxylic acid, 2,5-disulfoterephthalic acid, 2,5-diethoxyterephthalic acid, 2,5-diisopropylterephthalic acid, 2,3,5,6-tetrafluoroterephthalic acid, 2,3,5,6-tetrahydroxyterephthalic acid, 2,3,5,6-tetramethylterephthalic acid, 2,3,5,6-tetrakis(trifluoromethyl)terephthalic acid, benzene-1,2,3,4,5,6-hexacarboxylic acid, [1,1′-biphenyl]-4,4′-dicarboxylic acid, [1,1′:4,1″-terphenyl]-4,4″-dicarboxylic acid, and naphthalene-1,4-dicarboxylic acid.

8. The membrane according to Clause 7, wherein the ligand is selected from 2-aminoterephthalic acid or 2,5-dihydroxyterephthalic acid.

9. The membrane according to any one of the preceding clauses, wherein the substrate material is provided in the form of a mesh, a sheet or in the form of hollow fibers (e.g. porous alumina ceramic hollow fibers) and other arrangements that are obtainable by the folding of a mesh, a sheet and hollow fibers.

10. The membrane according to any one of the preceding clauses, wherein the polycrystalline metal-organic framework attached to the surface of the substrate material has a thickness of from 20 nm to 20 μm, such as from 800 nm to 20 μm, such as from 2 μm to 20 μm.

11. A method of using a polycrystalline metal-organic framework membrane as described in any one of Clauses 1 to 10 in a process of separating a fluid into a filtrate fluid and a retentate fluid, the process comprising the steps of:

(a) providing a fluid in need of separation to a polycrystalline metal-organic framework membrane as described in any one of Clauses 1 to 10;

(b) allowing or enabling a portion of the fluid to pass through the polycrystalline metal-organic framework membrane to provide a filtrate fluid and thereby providing a filtrate fluid; and

(c) collecting the filtrate fluid and retentate fluids.

12. The method according to Clause 11, wherein the fluid to be separated is selected from: a mixture of gases; an aqueous solution comprising one or more inorganic materials; an aqueous solution comprising one or more organic materials; an aqueous solution comprising one or more inorganic materials and one or more organic materials; a mixture of organic liquids; a mixture of one or more organic liquids and water; a mixture of one or more organic liquids and one or more organic materials; a mixture of one or more organic liquids and one or more inorganic materials; a mixture of one or more organic liquids, one or more organic materials and one or more inorganic materials; a mixture of water, one or more organic liquids and one or more organic materials; a mixture of water, one or more organic liquids and one or more inorganic materials; and a mixture of water, one or more organic liquids, one or more organic materials and one or more inorganic materials.

13. A method of forming a polycrystalline metal-organic framework membrane as described in any one of Clauses 1 to 10, the method comprising the steps of:
providing a seeded substrate having a surface seeded with seed crystals of the metal-organic framework, said seed crystals formed from a secondary building unit having the formula Ia or Ib and a ligand selected from fumaric acid, butynedioic acid, squaric acid, naphthalene-2,6-dicarboxylic acid, [2,2′-bipyridine]-5,5′-dicarboxylic acid, or a ligand having formula II, where formulae Ia, Ib and II are as described in any one of Clauses 1 to 10; and
subjecting the seeded substrate to a first mother liquor comprising a solvent, a metal salt precursor and a ligand selected from fumaric acid, butynedioic acid, squaric acid, naphthalene-2,6-dicarboxylic acid, [2,2′-bipyridine]-5,5′-dicarboxylic acid, or a ligand having formula as described above, for a first period of time under conditions sufficient to form a metal-organic framework membrane, wherein the metal salt precursor and the ligand are selected to form the same metal-organic framework as in the seed crystals.

14. The method of Clause 13, wherein the seeded substrate is formed by immersing a substrate having a surface in a second mother liquor that comprises a solvent, a metal salt precursor and a ligand selected from fumaric acid, butynedioic acid, squaric acid, naphthalene-2,6-dicarboxylic acid, [2,2′-bipyridine]-5,5′-dicarboxylic acid, or a ligand having formula II as described in any one of Clauses 1 to 10 for a second period of time to provide a seeded substrate having a surface seeded with seed crystals of the metal-organic framework, said seed crystals formed from a secondary building unit having the formula Ia or Ib and a ligand selected from fumaric acid, butynedioic acid, squaric acid, naphthalene-2,6-dicarboxylic acid, [2,2′-bipyridine]-5,5′-dicarboxylic acid, or a ligand having formula II, where formulae Ia, Ib and II are as described in any one of Clauses 1 to 10.

### DRAWINGS

Certain embodiments of the present disclosure are described more fully hereinafter with reference to the accompanying drawings.

FIG. 1. (a) PXRD patterns of simulated and experimental RE-MOFs. (b) The UiO-66-type topology.

FIG. 2. (a) N₂ sorption isotherms of RE-MOF (Y, Dy, Er, Gd and Sm) at 77 K (filled, adsorption; empty, desorption). (b) The pore size distribution of Sm-DOBDC calculated based on N₂ sorption data.

FIG. 3. (a) TGA curve of Sm-DOBDC. (b) Relative crystallinity of Sm-DOBDC after soaking in water/ethanol solutions for 7 days.

FIG. 4. SEM images of the alumina hollow fiber (a-b), seed layer (c-d), and membrane layer (e-f). (g) EDX elemental mapping of the cross-sectional area of the membrane shown in (f). (h) From left to right, PXRD patterns of Al₂O₃ substrate, Sm-DOBDC powder, and Sm-DOBDC membrane.

FIG. 5. Schematic diagram of the pervaporation set-up used in Example 2.

FIG. 6. (a) The membrane separation performance of 5 wt. % water/alcohol mixtures versus alcohol kinetic diameters (MeOH: methanol, EtOH: ethanol, NPA: 1-propanol, IPA: 2-propanol). (b) Effect of water content on membrane performance in separating water/ethanol mixtures. (c) Long-term stability of one Sm-DOBDC membrane in continuingly separating various water/alcohol mixtures (A: 5 wt. % water/2-propanol, B: 5 wt. % water/1-propanol, C: 5 wt. % water/methanol, D: immersion in 5 wt. % water/ethanol, E: 5 wt. % water/ethanol, F: 2 wt. % water/ethanol, G: 8 wt. % water/ethanol, H: 5 wt. % water/ethanol). (d) Effect of feed temperature on the dehydration performance of Sm-DOBDC membrane using 5 wt. % water/ethanol as the feed. Note that a freshly prepared Sm-DOBDC membrane was used in this test, accounting for the performance variation at 298 K compared to the result in (b).

FIG. 7. Water, methanol, and ethanol sorption isotherms of Sm-DOBDC at 293 K (filled, adsorption; empty, desorption).

FIG. 8. (a) Scheme of the in-situ healing process (circles represent membrane defects; diamonds represent newly grown Sm-DOBDC crystals during healing process). Top and cross-sectional FESEM images of the pure Al₂O₃ substrate treated with the supernatant generated during the Sm-DOBDC membrane preparation process under different time: (b, c) 21.7 h; (d, e) 25 h; (f, g) 35 h.

FIG. 9. The SEM images of the membrane before (a, c) and after (b, d) healing process (some intercrystal gaps are marked with circles). (e) The membrane separation performance using 5 wt. % ethanol/water feed before and after healing.

FIG. 10. FESEM images of DOBDC-based rare-earth MOFs: (a) Dy, (b) Er, (c) Gd, (d) Sm, and (e) Y.

FIG. 11. (a) CO₂ sorption isotherm of Sm-DOBDC at 273 K (filled, adsorption; empty, desorption). (b) The pore size distribution of Sm-DOBDC calculated based on CO₂ sorption data.

FIG. 12. SEM images of Sm-DOBDC membranes under different conditions: (a) as-prepared, (b) soaking in 5 wt. % water/methanol solution at 25° C. for 3 days, (c) soaking in 5 wt. % water/methanol solution at 25° C. for 7 days, (d) soaking in 5 wt. % water/ethanol solution at 50° C. for 1 day, (e) soaking in 5 wt. % water/ethanol solution at 25° C. for 3 days, and (f) soaking in 5 wt. % water/ethanol solution at 25° C. for 7 days.

FIG. 13. The dehydration performance of selected membranes toward 5 wt. % water/ethanol under identical testing conditions as compared to those previously reported: (a) data at 298 K, (b) data at 323 K.

FIG. 14. Top (a-c) and cross sectional (d-f) FESEM images of the Al₂O₃ substrate treated with the supernatant generated during the Zr-DOBDC membrane preparation process under different time: (a, d) 21.7 h; (b, e) 25 h; (c, f) 35 h.

FIG. 15. (a) Schematic diagram illustrating the preparation process of polycrystalline UiO-66-NH₂ membrane supported on flexible carbon cloth. (b) The tetrahedral cage (left), octahedral cage (middle), and 3D structure of UiO-66-NH₂ (right).

FIG. 16. XRD patterns of the simulated UiO-66(Zr)—NH₂ (a), carboxylated carbon cloth with UiO-66(Zr)—NH₂ seeds (b), UiO-66(Zr)—NH₂ powder collected from membrane synthesis solution (c), UiO-66(Zr)—NH₂ membrane prepared on carboxylated carbon cloth (d), and pristine carbon cloth (e).

FIG. 17. SEM images of the carbon cloth (a), the seed layer (b), and fully grown UiO-66(Zr)—NH₂ membrane (c: front view; d: cross-sectional view; e and f: cross-sectional EDS elemental scanning of Zr.

FIG. 18. (a) N₂ sorption isotherms at 77 K (closed, adsorption; open, desorption) and pore size distributions of the UiO-66(Zr)—NH₂ crystals before and after healing. (b) Single gas permeation and ideal selectivity of healed UiO-66(Zr)—NH₂ membrane (line in insert figure indicates the Knudsen diffusion selectivity of H₂ over other gases). The single gas permeation tests were performed at 22° C. under a transmembrane pressure of 1.0 bar. (c) Separation performance of UiO-66(Zr)—NH₂ membrane for NaCl aqueous solution (0.2 wt. %) under a transmembrane pressure of 3.0 bar before and after membrane healing. (d) Separation performance of UiO-66(Zr)—NH₂ membrane for multivalent ion aqueous solutions.

FIG. 19. Separation performance of the prepared UiO-66(Zr)—NH₂ membrane for the removal of dyes in dichloromethane and methanol solutions. UV-Vis absorption spectra of different dye solutions of (a, b) MB (in dichloromethane), (c, d) OR (in dichloromethane), (e, f) NR (in dichloromethane), (g, h) NR (in methanol) before filtration and after filtration through UiO-66(Zr)—NH₂ membranes. Photographs of the different dye solutions before and after filtration are shown in illustration.

FIG. 20. (a) Scheme for the membrane bending test. (b) Relationship between membrane bending-angle and the separation performance toward NaCl aqueous solution (0.2 wt. %). (c) Relationship between membrane bending-angle and the separation performance toward NR (100 ppm) in dichloromethane solution. Optical microscope and SEM images of UiO-66(Zr)—NH₂ membranes before (d, e, f) and after (g, h. i) bending tests.

FIG. 21. SEM images of the UiO-66(Zr)—NH₂ membranes obtained by two-step growth method with pristine carbon cloth as the substrate (a, b), and direct growth method with carboxylated carbon cloth as the substrate (c, d).

FIG. 22. SEM images of the UiO-66(Zr) membranes obtained by direct growth method using BDC as ligand: (a, b) with pristine carbon cloth as the substrate; (c, d) with carboxylated carbon cloth as the substrate.

FIG. 23. SEM images of the UiO-66(Zr) membranes obtained by two-step growth method using BDC as ligand: (a, b) with pristine carbon cloth as the substrate; (c, d) with carboxylated carbon cloth as the substrate.

FIG. 24. Schematic diagram of the apparatus for organic solvent nanofiltration as elaborated in Example 6.

FIG. 25. XRD patterns of UiO-66(Zr)—NH₂ crystals in different solutions: from bottom to top are NaCl aqueous solution (0.20 wt. %), aqueous solutions with pH values of 1, 3, 5, 7, 9, 11, 13, and pure dichloromethane, respectively.

### DESCRIPTION

It has been surprisingly found that the current invention provides superior separation characteristics over a wide range or solvent systems and conditions, while also retaining its chemical stability.

Thus, there is provided a polycrystalline metal-organic framework membrane comprising:
a substrate material having a surface; and
a polycrystalline metal-organic framework attached to the surface of the substrate material, wherein the polycrystalline metal-organic framework is formed from:
a secondary building unit having the formula Ia or Ib:

M₆O₄(OH)₄ Ia,
where M is selected from Zr, Hf, and Ti; or

M′₆(OH)₈ Ib
where M′ is selected from Sm, Y, Dy, Er, Gd, and Ce; and
a ligand selected from fumaric acid, butynedioic acid, squaric acid, naphthalene-2,6-dicarboxylic acid, [2,2′-bipyridine]-5,5′-dicarboxylic acid, or a ligand having formula II:

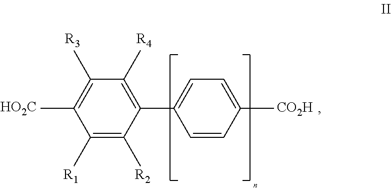

where:

R₁ is selected from H, halo, OR₅ₐ, SR_{5b}, C₁ to C₅ alkyl, NO₂, NR_{5c}R_{5d}, SO₃H, CF₃, or CO₂H;

R₂ is selected from H, halo, OR₆ₐ, SR_{6b}, C₁ to C₅ alkyl, NO₂, NR_{6c}R_{6d}, SO₃H, CF₃ or CO₂H;

R₃ is selected from H, halo, OR₇ₐ, SR_{7b}, C₁ to C₅ alkyl, NO₂, NR_{7c}R_{7d}, SO₃H, CF₃, or CO₂H;

R₄ is selected from H, halo, OR₈ₐ, SR_{8b}, C₁ to C₅ alkyl, NO₂, NR_{8c}R_{8d}, SO₃H, CF₃ or CO₂H;

R_{5a-5d}, R_{6a-6d}, R_{7a-7d}, R_{7a-7d} are each independently selected from H or C₁ to C₅ alkyl; or

R₁ and R₂ or R₃ and R₄ form, together with the carbon atoms to which they are attached to a C aromatic ring; and n is 0, 1 or 2.

In embodiments herein, the word “comprising” may be interpreted as requiring the features mentioned, but not limiting the presence of other features. Alternatively, the word “comprising” may also relate to the situation where only the components/features listed are intended to be present (e.g. the word “comprising” may be replaced by the phrases “consists of” or “consists essentially of”). It is explicitly contemplated that both the broader and narrower interpretations can be applied to all aspects and embodiments of the present invention. In other words, the word “comprising” and synonyms thereof may be replaced by the phrase “consisting of” or the phrase “consists essentially of” or synonyms thereof and vice versa.

When used herein, the term “secondary building unit” (SBU) refers to a molecular complex or a cluster entity in which ligand coordination modes and metal coordination environments can be utilized in the transformation of these fragments into extended porous networks using polytopic linkers. As such an SBU is intended to take its ordinary meaning in the art to which ligands are attached via their carboxylate groups.

Unless otherwise stated, the term “alkyl” refers to an unbranched or branched, cyclic, saturated or unsaturated (so forming, for example, an alkenyl or alkynyl) hydrocarbyl radical, which may be substituted or unsubstituted (with, for example, one or more halo atoms). Where the term “alkyl” refers to an acyclic group, it is preferably C₁₋₅ alkyl (such as ethyl, propyl, (e.g. n-propyl or isopropyl), butyl (e.g. branched or unbranched butyl), pentyl or, more preferably, methyl). Where the term “alkyl” is a cyclic group (which may be where the group “cycloalkyl” is specified), it is preferably C₃₋₅ cycloalkyl and, more preferably, C₅ cycloalkyl.

The term “halogen”, when used herein, includes fluorine, chlorine, bromine and iodine.

The substrate material may be formed from any suitable material, which includes, but is not limited to, a polymer, a ceramic (e.g. alumina), a carbon film/cloth, a metal and a metal oxide. The substrate can be in any suitable form, which include, but is not limited to, meshes, sheets and hollow fibers (e.g. porous alumina ceramic hollow fibers), plus other forms that can be obtained by the folding of these primary forms.

Examples of substrates in the form of sheets include, but are not limited to, polymer film and carbon film/cloth. Meshes may include, but are not limited to, metal meshes and metal oxide meshes. Hollow fiber structures that may be mentioned herein include, but are not limited to ceramics (e.g. alumina) and polymer films.

It is noted that certain substrates (e.g. carbon films/cloths or stainless steel meshes) can be functionalized by carboxylation or amination, which can facilitate the growth of crystal seeds. In addition, the flexibility of carbon films/cloths as substrates can offer good mechanical properties to the resultant membranes.

Examples of polymers that may be used as substrates include, but are not limited to, polyethyleneimine (PEI), polyethersulfone (PES), polyvinylidene fluoride (PVDF), polyetherimide (Ultem™ 1000), poly(ether-block-amide) (PEBA), polydimethylsiloxane (PDMS), poly(amic acid), polybenzimidazole (PBI), Pebax, Matrimid™, 6FDA-DAM, 6FDA/BPDA-DAM, poly(amide-imide), polydopamine (PDA), poly tetra fluoroethylene (PTFE), and combinations thereof.

In certain embodiments that may be mentioned herein, when the substrate material is alumina, then the secondary building unit may have formula Ia, where M is Zr or, more particularly, Hf or Ti or the secondary building unit has formula Ib. For example, when the substrate material is alumina, then the secondary building unit may have formula Ib.

While the polycrystalline metal-organic frameworks mentioned herein may take any suitable form, in embodiments mentioned herein, the polycrystalline metal-organic framework may be one that is a UiO-66-type metal-organic framework.

Any suitable ligand (or compatible mixture of ligands) may be used to form the polycrystalline metal-organic frameworks disclosed herein. As noted above, the ligand may be selected from (one or more of) fumaric acid, butynedioic acid, squaric acid, naphthalene-2,6-dicarboxylic acid, [2,2′-bipyridine]-5,5′-dicarboxylic acid, and a ligand having formula II:

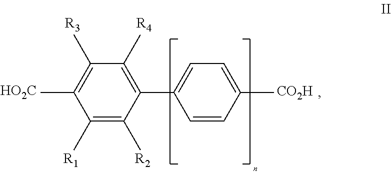

where:

R₁ is selected from H, halo, OR₅ₐ, SR_{5b}, C₁ to C₅ alkyl, NO₂, NR_{5c}R_{5d}, SO₃H, CF₃, or CO₂H;

R₂ is selected from H, halo, OR₆ₐ, SR_{6b}, C₁ to C₅ alkyl, NO₂, NR_{6c}R_{6d}, SO₃H, CF₃ or CO₂H;

R₃ is selected from H, halo, OR₇ₐ, SR_{7b}, C₁ to C₅ alkyl, NO₂, NR_{7c}R_{7d}, SO₃H, CF₃, or CO₂H;

R₄ is selected from H, halo, OR₈ₐ, SR_{8b}, C₁ to C₅ alkyl, NO₂, NR_{8c}R_{8d}, SO₃H, CF₃ or CO₂H;

R_{5a-5d}, R_{6a-6d}, R_{7a-7d}, R_{7a-7d} are each independently selected from H or C₁ to C₅ alkyl

In embodiments where the ligand has formula II, then,

R₁ may be selected from H, halo, OR₅ₐ, SR_{5b}, C₁ to C₅ alkyl, NO₂, NR_{5c}R_{5d}, SO₃H, CF₃, or CO₂H;

R₂ may be selected from H, OR₆ₐ, SR_{6b}, CF₃ or CO₂H;

R₃ may be selected from H, halo, OR₇ₐ, SR_{7b}, C₁ to C₅ alkyl, NR_{7c}R_{7d}, SO₃H, CF₃, or CO₂H; and

R₄ may be selected from H, F, OR₈ₐ, SR_{8b}, CF₃ or CO₂H.

In embodiments of formula II where two or more of R₁ to R₄ are not H, then the non-H substituents may be identical to each other. For example, R₁ and R₃ are both OH (with R₂ and R₄ being H), or R₁ and R₂ are both NH₂ (with R₃ and R₄ being H) etcetera.

In additional or alternative embodiments of the invention, n may be 0 or 1. For example n may be 0. As will be appreciated, when n is 1, the resulting pore size obtained will be larger than obtained when n is 0, but smaller than when n is 2. As such, varying n in the ligands of formula II allows one to vary the size of the pores in the resulting polycrystalline metal-organic framework and overall product. This allows one to tailor the product to the desired porosity to enable it to work for a specific application.

In addition, when the ligands used in the polycrystalline metal-organic frameworks described herein include reactive groups (e.g. —NH₂ groups), then the polycrystalline metal-organic framework may also be further functionalised to enable it to be used in further applications, such as a gas sensor, oil/water separation, photocatalysis, membrane reactors, and so on. Furthermore, when the ligands used in the polycrystalline metal-organic frameworks described herein include reactive groups, then the membrane can even be used as a reactive layer for the further growth of other membrane layers (J. Membr. Sci. 2019, 573, 97-106).

Specific ligands that may be used to make the polycrystalline metal-organic frameworks described herein include, but are not limited to fumaric acid, butynedioic acid, squaric acid, naphthalene-2,6-dicarboxylic acid, [2,2′-bipyridine]-5,5′-dicarboxylic acid, terephthalic acid, 2-fluoroterephthalic acid, 2-chloroterephthalic acid, 2-bromoterephthalic acid, 2-iodoterephthalic acid, 2-hydroxyterephthalic acid, 2-mercaptoterephthalic acid, 2-methylterephthalic acid, 2-nitroterephthalic acid, 2-aminoterephthalic acid, 2-sulfoterephthalic acid, 2-(trifluoromethyl)terephthalic acid, benzene-1,2,4-tricarboxylic acid, 2,3-dihydroxyterephthalic acid, 2,3-dimercaptoterephthalic acid, 2,5-difluoroterephthalic acid, 2,5-dichloroterephthalic acid, 2,5-dibromoterephthalic acid, 2,5-diiodoterephthalic acid, 2,5-terephthalic acid, 2,5-dihydroxyterephthalic acid, 2,5-dimercaptoterephthalic acid, 2,5-dimethylterephthalic acid, 2,5-diaminoterephthalic acid, 2,5-bis(trifluoromethyl)terephthalic acid, 2,5-diemthoxyterephthalic acid, benzene-1,2,4,5-tetracarboxylic acid, 2,5-disulfoterephthalic acid, 2,5-diethoxyterephthalic acid, 2,5-diisopropylterephthalic acid, 2,3,5,6-tetrafluoroterephthalic acid, 2,3,5,6-tetrahydroxyterephthalic acid, 2,3,5,6-tetramethylterephthalic acid, 2,3,5,6-tetrakis(trifluoromethyl)terephthalic acid, benzene-1,2,3,4,5,6-hexacarboxylic acid, [1,1′-biphenyl]-4,4′-dicarboxylic acid, [1,1′:4,1″-terphenyl]-4,4″-dicarboxylic acid, and naphthalene-1,4-dicarboxylic acid. In particular embodiments that may be mentioned herein, the ligand may be selected from 2-aminoterephthalic acid or 2,5-dihydroxyterephthalic acid. For example, the ligand may be 2-aminoterephthalic acid.

As will be appreciated, the polycrystalline metal-organic framework attached to the surface of the substrate material will result in a layer on top of the substrate material that will have a thickness. The thickness of this layer may be from 20 nm to 20 μm, such as from 800 nm to 20 μm, such as from 2 μm to 20 μm. In particular embodiments that may be mentioned herein, the thickness may be less than or equal to 500 nm, such as from 20 to 500 nm, such as from 50 to 499 nm, such as from 100 to 400 nm, such as from 200 to 300 nm.

For the avoidance of doubt, it is explicitly contemplated that where a number of numerical ranges related to the same feature are cited herein, that the end points for each range are intended to be combined in any order to provide further contemplated (and implicitly disclosed) ranges. Thus, in relation to the above related numerical ranges, there is disclosed:

from 20 nm to 50 nm, from 20 nm to 100 nm, from 20 nm to 200 nm, from 20 nm to 300 nm, from 20 nm to 400 nm, from 20 nm to 499 nm, from 20 nm to 500 nm, from 20 nm to 800 nm, from 20 nm to 2 μm, from 20 nm to 20 μm;

from 50 nm to 100 nm, from 50 nm to 200 nm, from 50 nm to 300 nm, from 50 nm to 400 nm, from 50 nm to 499 nm, from 50 nm to 500 nm, from 50 nm to 800 nm, from 50 nm to 2 am, from 50 nm to 20 μm;

from 100 nm to 200 nm, from 100 nm to 300 nm, from 100 nm to 400 nm, from 100 nm to 499 nm, from 100 nm to 500 nm, from 100 nm to 800 nm, from 100 nm to 2 μm, from 100 nm to 20 μm;

from 200 nm to 300 nm, from 200 nm to 400 nm, from 200 nm to 499 nm, from 200 nm to 500 nm, from 200 nm to 800 nm, from 200 nm to 2 μm, from 200 nm to 20 μm;

from 300 nm to 400 nm, from 300 nm to 499 nm, from 200 nm to 500 nm, from 300 nm to 800 nm, from 300 nm to 2 μm, from 300 nm to 20 μm;

from 400 nm to 499 nm, from 400 nm to 500 nm, from 400 nm to 800 nm, from 400 nm to 2 μm, from 400 nm to 20 μm;

from 499 nm to 500 nm, from 499 nm to 800 nm, from 499 nm to 2 μm, from 499 nm to 20 μm;

from 500 nm to 800 nm, from 500 nm to 2 μm, from 500 nm to 20 μm; and

from 800 nm to 2 μm, from 800 nm to 20 μm.

Any of the ranges mentioned above may be applied to any combination of embodiments listed herein unless otherwise specified.

In particular examples discussed herein, the thickness of the UiO-66(Zr) membranes fabricated on the carbon cloth and alumina hollow fiber were 800 nm and 3.5 μm, respectively.

Furthermore, the thickness of a rare earth membrane is about 20 μm.

When used herein the term “carbon cloth” may also cover carbon films.

The thickness of MOF membranes with a secondary building unit having the formula Ia fabricated on the carbon cloth and alumina hollow fiber in certain examples herein were 800 nm and 3.5 μm, respectively. Besides, the thickness of the MOF membranes with a secondary building unit having the formula Ib on the alumina hollow fiber was about 20 μm. Therefore, the thicknesses of polycrystalline metal-organic framework membranes mentioned in examples herein are 800 nm to 20 μm.

These thicknesses mentioned directly above may be reduced through changes in the method of manufacture and it may be desired to achieve a thickness of less than 500 nm, for example, from 200 to 300 nm.

The membranes described above may have a broad utility in the separation of fluids and materials within said fluids. Thus there is also disclosed a method of using a polycrystalline metal-organic framework membrane as described herein in a process of separating a fluid into a filtrate fluid and a retentate fluid, the process comprising the steps of:

(a) providing a fluid in need of separation to a polycrystalline metal-organic framework membrane as described herein;

(b) allowing or enabling a portion of the fluid to pass through the polycrystalline metal-organic framework membrane to provide a filtrate fluid and thereby providing a filtrate fluid; and

(c) collecting the filtrate fluid and retentate fluids.

There are multiple possible separations where the current invention may be beneficial. For example, the fluid to be separated may be selected from: a mixture of gases; an aqueous solution comprising one or more inorganic materials; an aqueous solution comprising one or more organic materials; an aqueous solution comprising one or more inorganic materials and one or more organic materials; a mixture of organic liquids; a mixture of one or more organic liquids and water; a mixture of one or more organic liquids and one or more organic materials; a mixture of one or more organic liquids and one or more inorganic materials; a mixture of one or more organic liquids, one or more organic materials and one or more inorganic materials; a mixture of water, one or more organic liquids and one or more organic materials; a mixture of water, one or more organic liquids and one or more inorganic materials; and a mixture of water, one or more organic liquids, one or more organic materials and one or more inorganic materials.

Without wishing to be bound by theory, metal-organic framework membranes with hydrophilic ligands may have good water-stability and so may be useful in the separation of mixtures involving water, such as desalination, the removal of metals from wastewater, alcohol dehydration, etc.

Additionally or alternatively, metal-organic framework membranes having ligands endowed with reactive groups (e.g. —NH₂ groups) may also be further functionalised to enable them to be used in further applications, such as a gas sensor, oil/water separation, photocatalysis, membrane reactors, and so on.

Particular applications that may be mentioned herein for the membranes include, but are not limited to: separation of organic/water mixtures or organic systems; desalination and wastewater purification (i.e. the removal of ions or dyes from wastewater, known as organic solvent nanofiltration); and gas separation. The examples below provide detailed descriptions and results for various membranes of the current invention applied to these technologies. As will be appreciated, application to the other separation methods can be extrapolated from the methods disclosed herein and would be readily achieved by a skilled person based upon the instruction provided in this document and their common knowledge.

The membranes described herein may be manufactured by any suitable method. A method of forming a polycrystalline metal-organic framework membrane as described herein comprises the steps of:
providing a seeded substrate having a surface seeded with seed crystals of the metal-organic framework, said seed crystals formed from a secondary building unit having the formula Ia or Ib and a ligand selected from fumaric acid, butynedioic acid, squaric acid, naphthalene-2,6-dicarboxylic acid, [2,2′-bipyridine]-5,5′-dicarboxylic acid, or a ligand having formula II, where formulae Ia, Ib and II are as described hereinbefore; and
subjecting the seeded substrate to a first mother liquor comprising a solvent, a metal salt precursor and a ligand selected from fumaric acid, butynedioic acid, squaric acid, naphthalene-2,6-dicarboxylic acid, [2,2′-bipyridine]-5,5′-dicarboxylic acid, or a ligand having formula II as described above, for a first period of time under conditions sufficient to form a metal-organic framework membrane, wherein the metal salt precursor and the ligand are selected to form the same metal-organic framework as in the seed crystals.

As will be appreciated, the seeded substrate used in the method outlined above is required for said method to work. The seeded substrate may be formed by immersing a substrate having a surface in a second mother liquor that comprises a solvent, a metal salt precursor and a ligand selected from fumaric acid, butynedioic acid, squaric acid, naphthalene-2,6-dicarboxylic acid, [2,2′-bipyridine]-5,5′-dicarboxylic acid, or a ligand having formula II as described hereinbefore for a second period of time to provide a seeded substrate having a surface seeded with seed crystals of the metal organic framework, said seed crystals formed from a secondary building unit having the formula Ia or Ib and a ligand selected from fumaric acid, butynedioic acid, squaric acid, naphthalene-2,6-dicarboxylic acid, [2,2′-bipyridine]-5,5′-dicarboxylic acid, or a ligand having formula II, where formulae Ia, Ib and II are as described hereinbefore.

Further aspects and embodiments of the invention are provided in the following non-limiting examples.

As described in Example 1, the membrane may be a polycrystalline rare-earth MOF membrane (Sm-DOBDC) supported on alumina hollow fiber. When separating 5 wt. % water/ethanol feed solution at 323 K, the Sm-DOBDC membrane exhibits high water stability with a total flux of 786.4±33.7 g·m⁻²·h⁻¹ and a 99.8±0.2 wt. % water concentration in permeate (separation factor: >9481) as shown in Example 2. Because of the reactive RE-containing building units, the Sm-DOBDC membrane can be healed in-situ by treating with reaction solution as shown in Example 3. As such, the membranes disclosed herein may generally have the ability to heal in-situ.

As described in Example 4, the membrane may be a continuous polycrystalline UiO-66(Zr)—NH₂ membrane supported on flexible carbon cloth substrate. The UiO-66(Zr)—NH₂ membrane possesses sub-micron thicknesses with a certain flexibility and angular bend tolerance up to 10°, as shown in Example 7. In addition, the UiO-66(Zr)—NH₂ membrane exhibits great separation performance in organic solvent nanofiltration (OSN) because of its excellent solvent resistance, with above 99.8% dye rejection and high flux for dichloromethane (ca. 0.17 kg m⁻² h⁻¹ bar⁻¹) as shown in Example 6. The UiO-66(Zr)—NH₂ membrane possesses low thickness (ca. 0.8 μm), high chemical stability (stable over a wide pH range from pH 1 to 11 as shown in Example 6), and low defect concentration arising from the optimized fabrication procedure of the membrane afford its excellent separation performance in both aqueous and organic solvent-based liquid separations. In particular, near complete rejection of dyes (MB, OR, and NR) and moderate permeance of organic solvents (0.175 kg m⁻² h⁻¹ bar⁻¹ for dichloromethane and 0.24 kg m⁻² h⁻¹ bar⁻¹ for methanol) as shown in Example 6 suggest promising applications of the membranes disclosed herein in solvent resistant nanofiltration, both those in the examples mentioned above and more generally.

### EXAMPLES

### Materials

Sm(NO₃)₃, Y(NO₃)₃, Dy(NO₃)₃, Er(NO₃)₃, and Gd(NO₃)₃ were purchased from Sinopharm Group Co. Ltd. 2-Fluorobenzoic acid (2-FBA) and 2,5-dihydroxy-1,4-benzenedicarboxylic acid (DOBDC) were obtained from Energy Chemical and Bepharm Ltd. N,N-dimethylformamide (DMF) and ethanol were supplied by Avantor Performance Materials, Inc. and VWR. Alumina ceramic tubes (O.D.: 1.96 mm, length: 50 mm, porosity: ca. 40%, average pore size: 1.6 μm) were provided by Nanjing Tech University and prepared in accordance with reported procedures (ACS Appl. Mater. Interfaces, 9 (2017) 22268-22277).

### Characterization

The morphology of Sm, Y, Dy, Er, and Gd-DOBDC MOF crystals and Al₂O₃ hollow fiber membranes were observed by field emission scanning electron microscopy (FESEM, JSM-7610F, JEOL). The crystal phases were measured by powder X-ray diffraction (PXRD, MiniFlex 600, Rigaku) equipped with a Cu sealed tube (λ=0.154178 nm) with a scan rate of 0.04 deg·s⁻¹ in the 2θ range of 5-50°. The original sample (20 mg) with the strongest peak intensity at 7.4° and 8.1° was denoted as 100% crystallinity. The samples were processed by acid/basic solution with different pH value or ethanol aqueous solutions with different ratios of ethanol to water, and the relative crystallinity of samples was obtained by calculating the ratio of the peak intensity. N₂ sorption isotherms were measured using a Micromeritics ASAP 2020 surface area and pore size analyzer. Pore size distribution data were calculated from the N₂ adsorption isotherms at 77 K based on nonlocal density functional theory (NLDFT) model. Thermogravimetric analyses (TGA) were performed at a heating rate of 10° C./min using a Shimadzu DTG-60AH.

Preparation of Rare-Earth MOFs and their Characterisation

A series of rare-earth fcu MOFs (RE-MOFs, RE=Sm, Y, Dy, Er, Gd) based on 2,5-dihydroxy-1,4-benzenedicarboxylate (DOBDC) as the ligand and 2-fluorobenzoic acid (2-FBA) as the modulator and structure directing agent were prepared via a solvothermal method.

In a typical experiment, Sm(NO₃)₃.6H₂O and DOBDC were firstly dispersed into the DMF/ethanol mixing solution (V_{DMF}:Vₑₜₕₐₙₒₗ=2:1) under ultrasonic stirring, followed by adding 2-FBA and cultivating for 3 days at 105° C. A molar composition of 1 Sm(NO₃)₃: 1.5 DOBDC: 500 DMF/ethanol: 70 2-FBA was adopted. The resultant products were washed with DMF and ethanol, and then heated at 120° C. under vacuum overnight.

### Characterisation Results of RE-MOFs

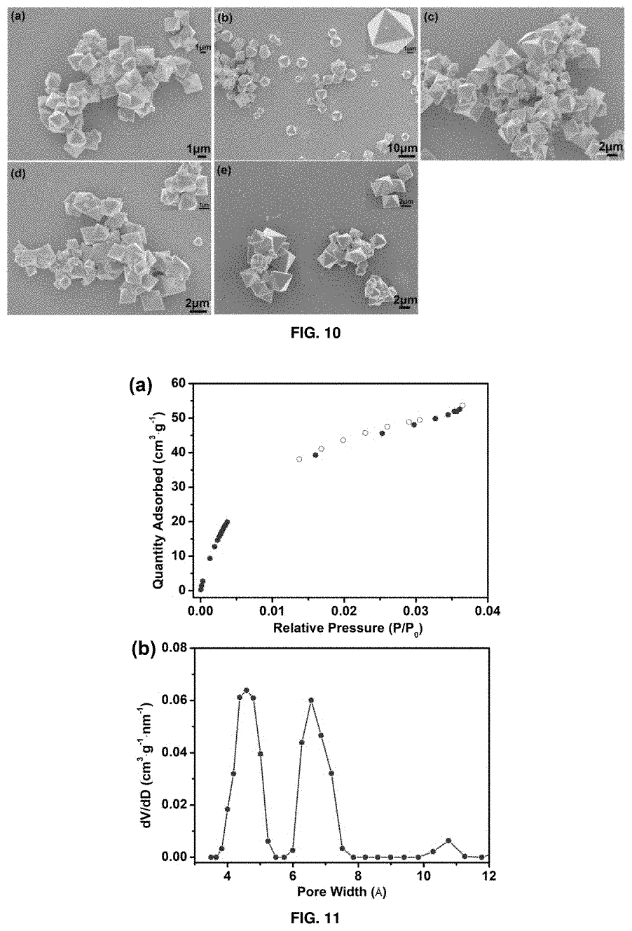

The synthesized Sm, Y, Dy, Er, and Gd-MOFs have clear facets with octahedral crystal sizes of about 2-3 μm based on SEM images (FIG. 10).

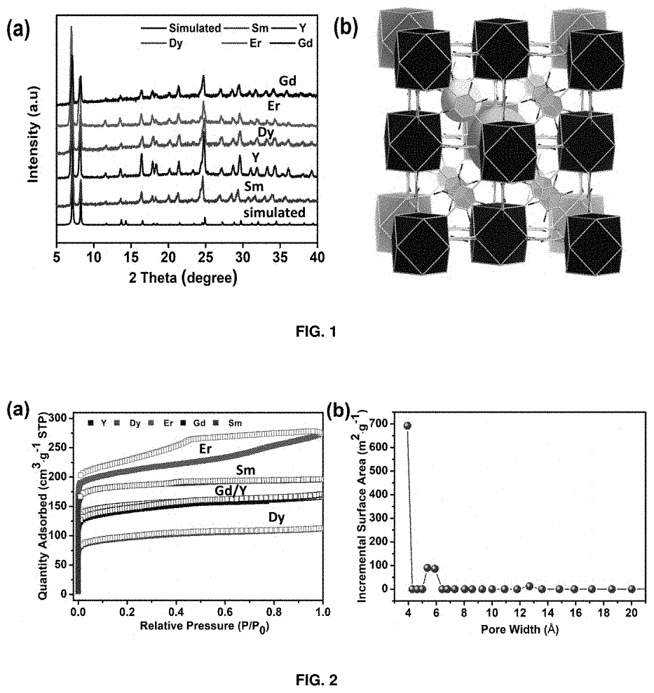

Their powder X-ray diffraction (PXRD) patterns (FIG. 1a) show well-defined diffraction peaks in close agreement with the simulated one based on the UiO-66 topology (FIG. 1b).

The activated RE-MOFs exhibit Type I N₂ sorption isotherms at 77 K, with Brunauer-Emmett-Teller (BET) surface areas in the range of 302-653 m²·g⁻¹ (FIG. 2a).

### Characterisation Results of Sm-DOBDC MOFs

Sm-DOBDC was chosen as the target membrane material because of its prominent BET surface area (520 m²·g⁻¹) and a mainly microporous structure.

The pore size distribution of Sm-DOBDC reflects significant microporosity at about 4 Å (FIG. 2b and FIG. 11). Notably, this pore size is smaller than that of UiO-66(Zr)—(OH)₂ (5.89 Å), which can be attributed to fewer defects in RE-MOFs. In theory, the smaller pore size of Sm-DOBDC is suitable for water/organics separation in which organic molecules with larger kinetics diameters (i.e., 4.5 Å for ethanol) can be blocked while water molecules (2.8 Å) are allowed to pass unhindered.

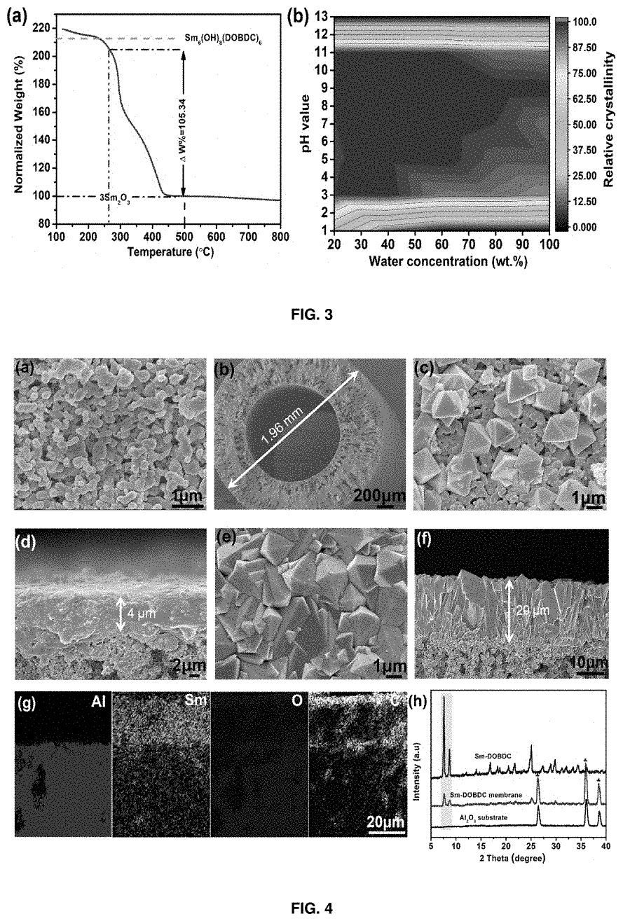

On the basis of previous studies, the weight loss of MOFs during thermogravimetric analyses (TGA) is inversely correlated with the defects of the MOFs. The theoretical TGA plateau for Sm₆(OH)₈(DOBDC)₆ after removing all the solvents should be at 212.8% when the weight of the end product, Sm₂O₃, is normalized to 100%. The theoretical ratio between DOBDC and the Sm₆ SBU should be 6 in an ideal framework, therefore the weight contribution per DOBDC linker would be 18.8% [(212.8%-100%)/6]. Based on the experimental TGA plateau of 205% in the obtained Sm-DOBDC after removing all the solvents, the real ratio between DOBDC and the Sm₆ SBU can be determined as 5.6 [(205%-100%)/18.8%] (FIG. 3a), equivalent to 6.7% of coordinative defects [(6-5.6)/6]. This ratio is even lower than that of the post-synthetically healed UiO-66(Zr)—(OH)₂ (16.7%), which can be attributed to the special coordination preference (12-connected RE-containing secondary building units) and low defect tolerance of the RE-SBUs.

### Water Stability of Sm-DOBDC MOFs

The stability of Sm-DOBDC was evaluated by soaking the crystals in water/ethanol solution for 7 days with various ratios of water (i.e., 0, 20, 40, 60, 80, and 100% of water) under different pH values (i.e., 1, 3, 5, 7, 9, 11, and 13), and checking the relative crystallinity based on the PXRD diffraction peak intensity at 26=7.0 and 8.1°.

The relative crystallinity of Sm-DOBDC is above 80% under a pH value range of 3-11 with various ethanol/water ratios, confirming the excellent chemical stability of Sm-DOBDC that is suitable for alcohol dehydration even under harsh conditions (FIG. 3b).

### Example 1: Preparation and Characterisation of Sm-DOBDC Membranes

Defect-free Sm-BOBDC membrane was fabricated on alumina hollow fibers by the secondary growth method.

### Preparation of Sm-DOBDC Membrane

The polycrystalline Sm-DOBDC membranes were fabricated on the outer surface of porous alumina ceramic hollow fibers by secondary growth synthesis. The alumina supports with both ends sealed were placed in a polytetrafluoroethylene holder, and immersed into a mother solution with a molar composition of 1 Sm(NO₃)₃: 1.5 DOBDC: 500 DMF/ethanol: 70 2-FBA.

The crystallization was executed at 105° C. for (1+3) days (in-situ growth: 1 day, secondary growth: 3 days) in a Teflon-lined stainless steel autoclave. Specifically, the cultivation was performed at 105° C. for 24 h (1 day) in a Teflon-lined stainless steel autoclave. In this cultivation Sm-DOBDC nanocrystals were seeded on the outer of the support. After cooling to room temperature, the seeded Sm-DOBDC membranes were thoroughly washed with DMF and ethanol for several times, followed by drying at room temperature overnight. The crystal seeds grown on the substrate were further integrated together to form a continuous and well-intergrown polycrystalline Sm-DOBDC membrane by secondary growth in the same mother solution conducted at 105° C. for 72 h (3 days). After cooling to room temperature, the obtained Sm-DOBDC membranes were sequentially washed with DMF and ethanol, then dried at room temperature overnight before further tests.

Characterisation of Seeded Sm-DOBDC Membranes after 1 Day In-Situ Growth

The seeded Sm-DOBDC membrane mainly comprises an amorphous membrane layer, and some Sm-DOBDC seeds were randomly deposited on the surface of the alumina substrate (FIG. 4c, d). A semi-continuous seed layer with large boundary voids could be clearly observed.

Due to the large crystal grains (2-3 μm), the thickness of the Sm-DOBDC seeding layer is approximately 4 μm.

The PXRD spectrum of the crystals collected from the bottom of the reaction container matches well with the simulated one (not included). These results suggest that the Sm-DOBDC crystals prefer homogeneous nucleation rather than membrane formation on alumina substrates because of the limited heterogeneous nucleation sites.

Characterisation of Sm-DOBDC Membranes after 3-Day Secondary Growth

After solvothermal synthesis at 105° C. for 72 h, well-intergrown polycrystalline Sm-DOBDC membranes without any visible cracks or pinholes were fabricated (FIGS. 4e, f, and h). The crystals grow adjacently to each other, with well-defined octahedral morphology. The crystal grain size increases until forming a continuous layer after secondary growth, indicating epitaxial growth of the previously nucleated Sm-DOBDC crystal seeds during the secondary growth process.

The thickness of Sm-DOBDC membrane was estimated to be about 20 μm, which is thicker than those of UiO-66 membranes (s 6 μm) while thinner than that of sod-ZMOF membrane (ca. 30 μm).

The energy-dispersive X-ray (EDX) images (FIG. 4g) reveal an obvious transition between the membrane layer and the substrate, suggesting a successful growth of a continuous Sm-DOBDC layer on the Al₂O₃ support.

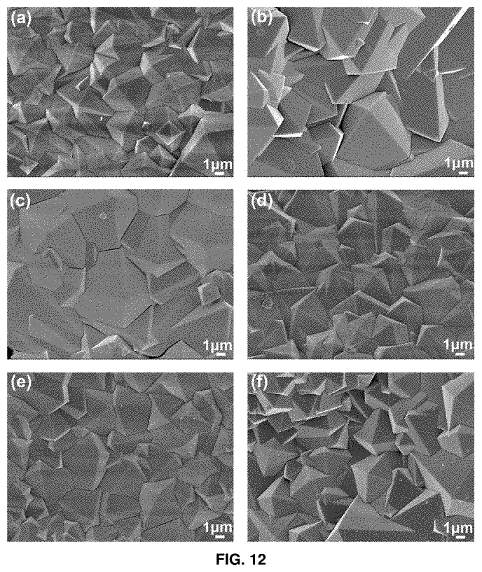

To further verify their water stability, Sm-DOBDC membranes were soaked in methanol/water and ethanol/water solutions for several days. After soaking, the crystal grains in the membranes were still closely arranged without any etching, indicating their high stability in alcohol/water solutions (FIG. 12).

### Example 2: Use of Sm-DOBDC Membrane for Pervaporation-Based Separation of Alcohol/Water Mixtures

The separation performance of the Sm-DOBDC membrane as prepared according to Example 1 was evaluated by pervaporation-based alcohol dehydration.

Experimental Set-Up and Calculations

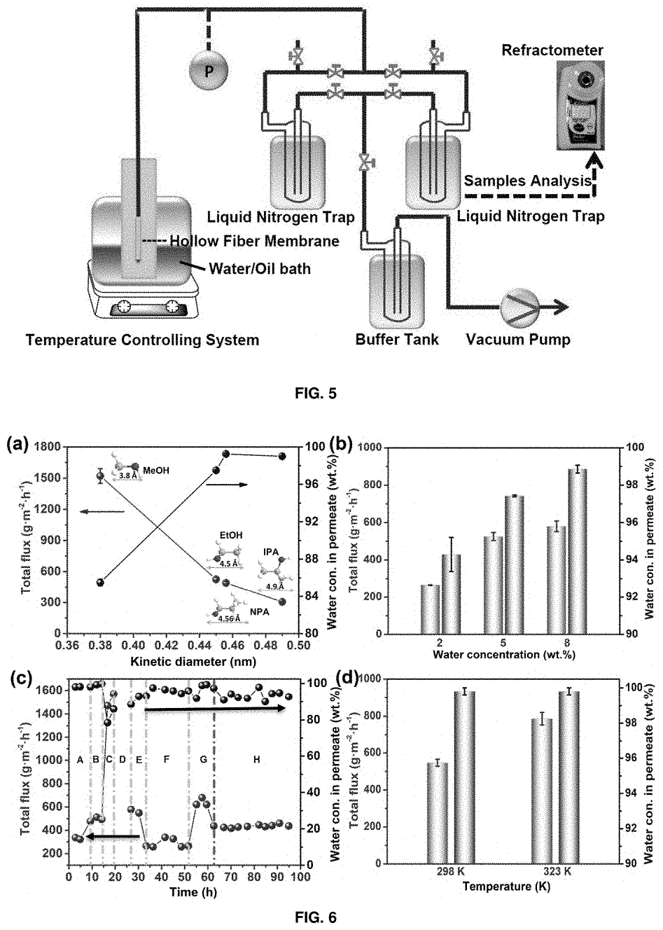

The performance of the membranes was evaluated via pervaporation for separating water from aqueous organics using a home-built set-up (FIG. 5). One end of the membrane was sealed with silicone and the other open end was assembled in the module. The effective length (ca. 25 mm) and diameter of the membrane were accurately measured. The Sm-DOBDC hollow fiber membrane was immersed in the as-prepared alcohol/water mixtures, and the pressure of the permeate side was maintained at about 250 Pa.

Before collecting samples, 10 min was given to the system for stabilization. The permeate vapour was collected with a cold trap equipped with liquid nitrogen. The alcohol concentration of the permeate side sample was estimated by a refractometer (PAL-RI, ATAGO). This was done by obtaining the relationships between known concentrations of methanol, ethanol, 1-propanol, and 2-propanol versus their refractive indexes, and then comparing the measured refractive index with the obtained relationships to determine the alcohol concentration of the permeate side sample. The permeate sample was collected for three times, and the average value was obtained.

The total permeation flux (F) was obtained by weighing the condensate of the cold trap (Equation (1)).

F=W/(A×Δt) Eq. (1)

where W refers to the mass of liquids collected from permeate (g); A is the effective membrane area (m²); and Δt is the duration of the sample collection (h).

The separation factor (a) is formulated as (Equation (2))

α=[Y_{water}/(1−Y_{water})]/[X_{water}/(1−X_{water})] Eq. (2)

where Y_{water} and X_{water} represent the mass fraction of water in the permeate and feed sides, respectively.

### Effect of Alcohol Kinematic Diameter on Flux and Water Concentration in Permeate

The measured fluxes appear to decrease with increasing kinetic diameters of the alcohols (FIG. 6a). For the 5 wt. % water/methanol feed solution, a total flux of 1520.8±69.9 g·m⁻²·h⁻¹ was obtained, compared to 525±21.2, 491±14.8, and 305.5±4.9 g·m⁻²·h⁻¹ for water/ethanol, water/1-propanol, and water/2-propanol mixtures, respectively. Because the pore size of Sm-DOBDC is about 4 Å, the membrane can theoretically exclude ethanol, 1-propanol, and 2-propanol, while allow the permeation of water and methanol.

The water concentration in permeate increases from 85.5±0.35 wt. % (for water/methanol mixture) to above 99 wt. % (for water/2-propanol and water/1-propanol mixtures), and the corresponding separation factors of water/methanol, ethanol, 2-propanol, and 1-propanol feed solutions are 112.0±3.2, 741.0±15, 2514.3±340, and 1881±145, respectively, supporting the molecular sieving separation mechanism.

### Effect of Water Content in Feed on Flux and Water Concentration in Permeate

When the feed concentration increases from 2 wt % water/ethanol to 8% wt % water/ethanol, more water molecules can favorably pass through the MOF layer, and the total flux increases from 265.8±1.4 to 580±28.3 g·m⁻²·h⁻¹ (FIG. 6b).

For 2, 5, and 8 wt. % water/ethanol feed solutions, the water concentrations in permeate are 94.29±0.91, 97.5±0.05, and 98.86±0.21 wt. % with the separation factors of 809.1±160, 741.0±15, and 997.2±160, respectively (FIG. 6b).

### Effect of Time on Water Concentration in Permeate

The long-term stability of the membrane was assessed within 95 h (FIG. 6c), and the water concentration in permeate achieves above 85 wt. % except for water/methanol feed solution. The permeate water concentration remains above 98 wt. % for 1-propanol and 2-propanol aqueous solutions.

The total flux is about 417.2-460.7 g·m⁻²·h⁻¹ within the time range of 62.6-94.9 h, demonstrating the long-term stability of the membrane in aqueous solutions.

### Effect of Feed Temperature on Ethanol Dehydration Performance

The temperature effect on the ethanol dehydration performance was evaluated using another freshly prepared Sm-DOBDC membrane. Due to the variation in membrane quality, this membrane exhibited even better separation performance for the dehydration of 5 wt. % water/ethanol feed solution, with a total flux of 546.7±18.3 g·m⁻²·h⁻¹ and a 99.8±0.2 wt. % water concentration in permeate being achieved at 298 K, equivalent to a separation factor of above 9481.

When the testing temperature was raised to 323 K, the total flux increased by 44% to 786.4±33.7 g·m⁻²·h⁻¹ due to accelerated molecular diffusion at higher temperatures; surprisingly, water concentration in the permeate remained unchanged (99.8±0.2 wt. %), suggesting an almost perfect molecular sieving separation mechanism (FIG. 6d). These results indicate that the Sm-DOBDC membrane can retain its excellent pervaporation performance for ethanol/water feed solutions even at high temperatures.

### Comparing Separation Performance of Sm-DOBDC Membrane Against Other Reported Polycrystalline MOF Membranes

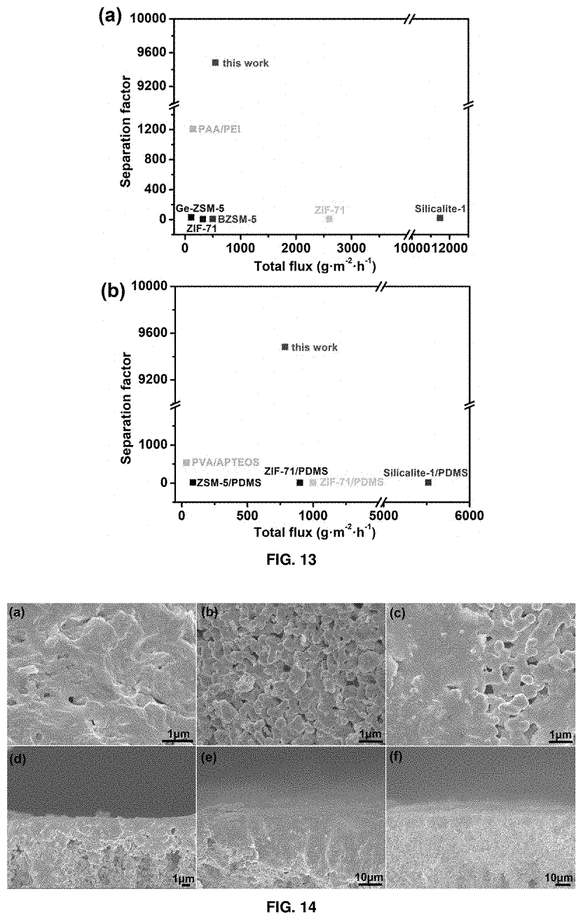

Compared with other polycrystalline MOF membranes reported previously such as UiO-, ZIF-, and MIL-membranes (FIG. 13, Table 1-3), the Sm-DOBDC membrane displays much higher separation factors and considerable total fluxes.

TABLE 1
Performance summary of membrane-based ethanol dehydration

| | | | Flux | Separation | |
|---|---|---|---|---|---|
| Feed mixture | | Temp. | (gm⁻² · | factor | |
| (mass ratio) | Membrane | (K) | h⁻¹) | (water/organic) | Ref. |
| EtOH/H₂O (95/5) | BZSM-5 | 298 | 500 | 8.2 | [1] |
| EtOH/H₂O (90/10) | UiO-66 | 303 | 640 | 4.3 | [2] |
| EtOH/H₂O (90/10) | UiO-66 | 343 | 3730 ± 120 | 55.8 ± 1.0 | [3] |
| EtOH/H₂O (95/5) | ZIF-71 | 298 | 322.18 | 6.07 | [4] |
| EtOH/H₂O | MIL-96 | 333 | 125 | 6 | [5] |
| (94.4/5.6) | | | | | |
| EtOH/H₂O (95/5) | ZIF-71 | 298 | 2600 | 6.88 | [6] |
| EtOH/H₂O (90/10) | CAU-10-H | 313 | 397 | 324 | [7] |
| EtOH/H₂O (90/10) | CAU-10-H | 338 | 493 | 148 | [7] |
| EtOH/H₂O (95/5) | ZIF-71/PDMS | 323 | −900 | 9.9 | [8] |
| EtOH/H₂O | MIL-53(AI)- | 313 | −900 | −13 | [9] |
| (92.5/7.5) | NH2/PVA | | | | |
| EtOH/H₂O (95/5) | Sm-DOBDC | 298 | 546.7 ± 18.3 | >9481 | this |
| | | | | | work |
| EtOH/H₂O (95/5) | Sm-DOBDC | 323 | 786.4 ± 33.7 | >9481 | this |
| | | | | | work |
| EtOH/H₂O (92/8) | Sm-DOBDC | 298 | 580 ± 28.3 | 997.2 ± 160 | this |
| | | | | | work |
| EtOH/H₂O (98/2) | Sm-DOBDC | 298 | 265.8 ± 1.4 | 809.1 ± 160 | this |
| | | | | | work |

TABLE 2
Performance summary of membrane-based methanol dehydration

| | | | Flux | Separation | |
|---|---|---|---|---|---|
| Feed mixture | | Temp. | (gm⁻² · | factor | |
| (mass ratio) | Membrane | (K) | h⁻¹) | (water/organic) | Ref. |
| MeOH/HO (95/5) | B-ZSM-5 | 333 | 900 | 8.4 | [10] |
| MeOH/H₂O | Pervap 2201 | 333 | 280 | ≈7.5 | [11] |
| (97.1/2.9) | | | | | |
| MeOH/H₂O (85/15) | Polyphenylsulfone | 333 | 33 | 11 | [12] |
| MeOH/H₂O (90/10) | UiO-66 | 323 | 1580 | 5.0 | [2] |
| MeOH/H₂O (95/5) | ZIF-71 | 298 | 394.64 | 21.38 | [4] |
| MeOH/H₂O (95/5) | ZIF-71/PDMS | 323 | — | 8.0 | [8] |
| MeOH/H₂O (95/5) | Sm-DOBDC | 298 | 1520.8 ± 69.9 | 112.0 ± 3.2 | this |
| | | | | | work |

TABLE 3
Performance summary of membrane-based propanol dehydration

| | | | Flux | Separation | |
|---|---|---|---|---|---|
| Feed mixture | | Temp. | (gm⁻² · | factor | |
| (mass ratio) | Membrane | (K) | h⁻¹) | (water/organic) | Ref. |
| IPA/H₂O (95/5) | B-ZSM-11 | 333 | 310 | 16 | [13] |
| IPA/H₂O (95/5) | B-ZSM-5 | 333 | 71 | 42 | [10] |
| IPA/H₂O (90/10) | UiO-66 | 343 | 4620 ± 90 | 689 ± 55 | [3] |
| IPA/H₂O (85/15) | ZIF-8/PBI | 333 | 103 | 1686 | [14] |
| IPA/H₂O (95/5) | ZIF-71/PDMS | 323 | — | 13.6 | [8] |
| IPA/H₂O (85/15) | ZIF-90/P84 | 333 | 114 | 385 | [15] |
| IPA/H₂O (90/10) | ZIF-8/PVA | 303 | 868 | 132 | [16] |
| IPA/H₂O (95/5) | Sm-DOBDC | 298 | 305.5 ± 4.95 | 1881 ± 145 | this |
| | | | | | work |

Note:
EtOH: ethanol;
MeOH: methanol;
IA: isopropanol;
PVA: poly(vinyl alcohol);
PDMS: Polydimethylsiloxane;
PBI: polybenzimidazole

[1] F. H. Saboor, S. N. Ashrafizadeh, H. Kazemian, Synthesis of BZSM-5 membranes using nano-zeolitic seeds: characterization and separation performance, Chem. Eng. Technol., 35 (2012) 743-753.
[2] M. Miyamoto, K. Hori, T. Goshima, N. Takaya, Y. Oumi, S. Uemiya, An organoselective zirconium-based metal-organic-framework UiO-66 membrane for pervaporation, Eur. J. Inorg. Chem., 2017 (2017) 2094-2099.
[3] X. Liu, C. Wang, B. Wang, K. Li, Novel organic-dehydration membranes prepared from zirconium metal-organic frameworks, Adv. Funct. Mater., 27 (2017) 1604311.
[4] X. Dong, Y. S. Lin, Synthesis of an organophilic ZIF-71 membrane for pervaporation solvent separation, Chem. Commun., 49 (2013) 1196-1198.
[5] Y. Hu, X. Dong, J. Nan, W. Jin, X. Ren, N. Xu, Y. M. Lee, Metal-organic framework membranes fabricated via reactive seeding, Chem. Commun., 47 (2011) 737-739.
[6] K. Huang, Q. Li, G. Liu, J. Shen, K. Guan, W. Jin, A ZIF-71 hollow fiber membrane fabricated by contra-diffusion, ACS Appl. Mater. Interfaces, 7 (2015) 16157-16160.
[7] H. Jin, K. Mo, F. Wen, Y. Li, Preparation and pervaporation performance of CAU-10-H MOF membranes, J. Membr. Sci., 577 (2019) 129-136.
[8] Y. Li, L. H. Wee, J. A. Martens, I. F. J. Vankelecom, ZIF-71 as a potential filler to prepare pervaporation membranes for bio-alcohol recovery, J. Mater. Chem. A, 2 (2014) 10034-10040.
[9] G. Wu, M. Jiang, T. Zhang, Z. Jia, Tunable pervaporation performance of modified MIL-53(Al)—NH₂/poly(vinyl alcohol) mixed matrix membranes, J. Membr. Sci., 507 (2016) 72-80.
[10] T. C. Bowen, H. Kalipcilar, J. L. Falconer, R. D. Noble, Pervaporation of organic/water mixtures through B-ZSM-5 zeolite membranes on monolith supports, J. Membr. Sci., 215 (2003) 235-247.
[11] D. Van Baelen, B. Van der Bruggen, K. Van den Dungen, J. Degreve, C. Vandecasteele, Pervaporation of water-alcohol mixtures and acetic acid-water mixtures, Chem. Eng. Sci., 60 (2005) 1583-1590.
[12] Y. Tang, N. Widjojo, G. M. Shi, T. S. Chung, M. Weber, C. Maletzko, Development of flat-sheet membranes for C1-C4 alcohols dehydration via pervaporation from sulfonated polyphenylsulfone (sPPSU), J. Membr. Sci., 415-416 (2012) 686-695.
[13] S. Li, V. A. Tuan, R. D. Noble, J. L. Falconer, ZSM-11 membranes: characterization and pervaporation performance, AlChE J., 48 (2002) 269-278.
[14] G. M. Shi, T. Yang, T. S. Chung, Polybenzimidazole (PBI)/zeolitic imidazolate frameworks (ZIF-8) mixed matrix membranes for pervaporation dehydration of alcohols, J. Membr. Sci., 415-416 (2012) 577-586.
[15] D. Hua, Y. K. Ong, Y. Wang, T. Yang, T. S. Chung, ZIF-90/P84 mixed matrix membranes for pervaporation dehydration of isopropanol, J. Membr. Sci., 453 (2014) 155-167.
[16] M. Amirilargani, B. Sadatnia, Poly(vinyl alcohol)/zeolitic imidazolate frameworks (ZIF-8) mixed matrix membranes for pervaporation dehydration of isopropanol, J. Membr. Sci., 469 (2014) 1-10.

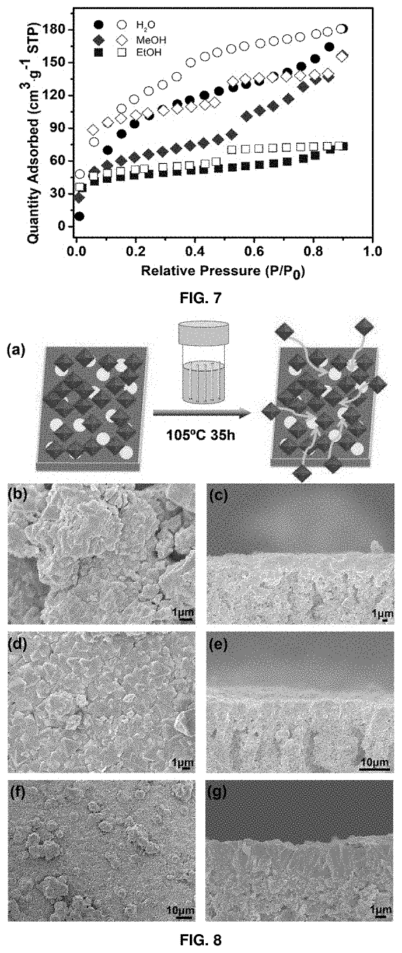

To further interpret the mechanism governing the excellent alcohol dehydration performance of the membrane, vapor sorption tests of water, methanol, and ethanol were performed at 293 K using a commercial instrument Quantachrome iQ3 (FIG. 7). The samples were activated before the tests. The fast water uptake at low pressures (P/P₀<0.1) surpassing those of methanol and ethanol can be attributed to the strong interaction between water molecules and Sm-DOBDC. At P/P₀=0.9, the adsorption capacities of water, methanol, and ethanol are 181, 156.7, and 73.5 cm³·g⁻¹, respectively. These results suggest that sorption may be a minor reason for the dehydration performance of the Sm-DOBDC membranes, while size-selective molecular sieving should be the dominating factor.

### Example 3: In-Situ Healing of Sm-DOBDC Membranes

The possibility of in-situ healing in Sm-DOBDC membranes was demonstrated to address defect formation during the operation of polycrystalline membranes.

### Procedure

A partially degraded Sm-DOBDC membrane was prepared by immersing a freshly prepared membrane into a 80 wt. % water/ethanol solution (pH=2) for a certain period of time (up to 35 h, please refer to the experimental details in the next paragraph) to mimic the possible membrane degradation during long-term operation under corrosive environments. In-situ healing was conducted by treating the partially degraded membrane at 105° C. with the supernatant generated during the Sm-DOBDC membrane preparation process (FIG. 8a).

To elaborate on the healing procedure, the degraded Sm-DOBDC membrane was placed into a 20 mL scintillation vial filled with the supernatant which can be repeatedly gathered in the membrane preparation or healing procedure. Then, the membrane was cultivated at 105° C. for 2100 min (35 h). Finally, the healed membrane was washed with DMF and ethanol for three times, respectively, followed by activation in the fume hood overnight.

To identify the appropriate healing time, pure Al₂O₃ hollow fiber was treated with the supernatant under different periods (21.7, 25, or 35 h). A continuous Sm-DOBDC layer could be formed at 35 h. Therefore, the in-situ healing process of defective Sm-DOBDC membranes was proceeded at 35 h (FIG. 8b-g).

### Effect of Healing on Membrane Morphology

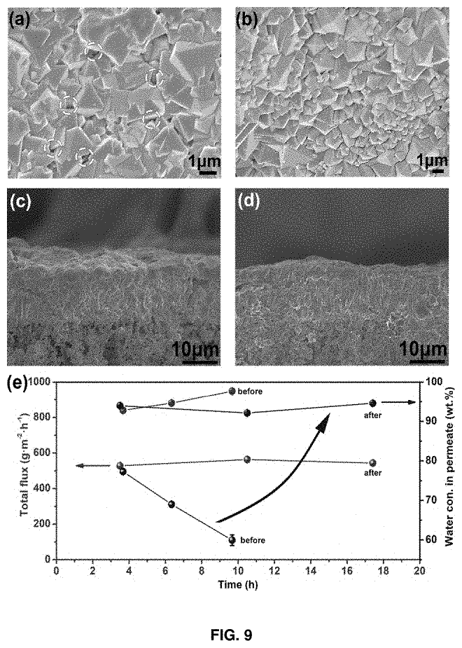

After the healing process, the apparent intercrystal gaps in the partially degraded Sm-DOBDC membrane were completely covered with newly grown small Sm-DOBDC crystals (FIG. 9a, b). The newly grown crystals have identical morphology with confined crystal growth mainly in the intercrystal gaps. Notably, there is not much change in the membrane thickness after the healing process (FIG. 9c, d).

### Effect of Healing on Separation Performance

The membrane performance was evaluated using 5 wt. % water/ethanol feed solution at 298 K based on the set-up described in Example 2. For the deteriorated Sm-DOBDC membrane, the total flux increased to 948.8±4.1 g·m⁻²·h⁻¹, and the permeation water concentration decreased to 60.0±1.3 wt. %. In contrast, the total flux of the healed membrane could reach 563.4±7.0 g·m⁻²·h⁻¹, and the permeate water concentration reverted to 94.6±0.2 wt. % even after 18 h of test, confirming that the healing treatment can effectively restore the separation performance (FIG. 9e).

Without wishing to be bound by theory, the healing mechanism can be explained as follows. Since there are many small nuclei in the solution adopted for the healing process, the Sm-DOBDC nanocrystals were gradually shaped and deposited on the surface of the defective Sm-DOBDC membranes after incubation at 105° C. for a period of time. Notably, the new Sm-DOBDC grains preferentially grew on the gaps and deficiencies of the membranes, without altering the thickness of the membranes in the chosen reaction time. Therefore, excellent separation performance was obtained in the healed Sm-DOBDC membrane.

### Comparative Example: Healing of Zr-DOBDC Membranes

In order to further illustrate the unique in-situ healing property of Sm-DOBDC membrane, a similar healing procedure was applied to Zr-MOF membranes. Briefly, pure Al₂O₃ substrate was treated with the supernatant generated during the preparation of Zr-DOBDC membrane (aka UiO-66-(OH)₂ membrane, ACS Appl. Mater. Interfaces 2017, 9, 37848-37855).

At 21.7, 25, and 35 h, the substrate surface was all deposited with amorphous solid particles, and no continuous Zr-DOBDC polycrystalline membrane layers could be found (FIG. 14). The result suggests that it is challenging to remove the defects in Zr-DOBDC membrane by in-situ healing because of the extremely harsh environment for the heterogeneous nucleation and growth of Zr-MOF membranes. In contrast, Sm-DOBDC membrane can be healed in-situ by treating with reaction solution due to the reactive RE-containing building units.

### Example 4: Preparation and Characterization of Polycrystalline UiO-66-NH₂ Membrane

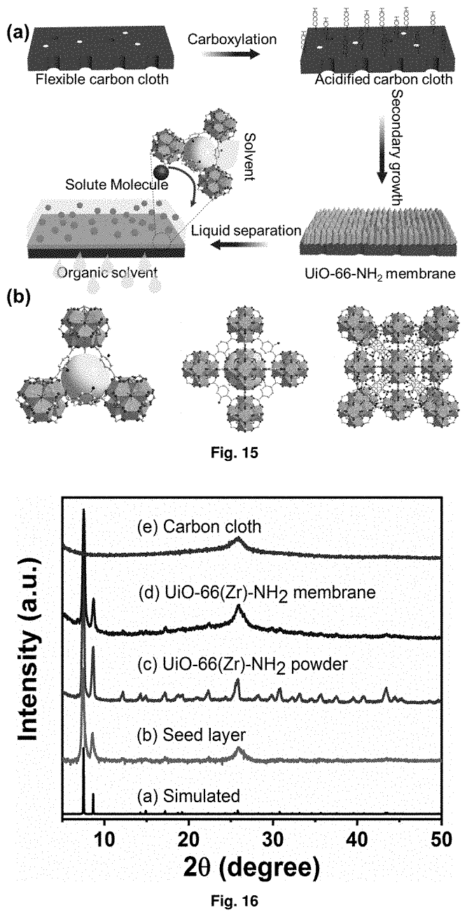

A continuous polycrystalline UiO-66(Zr)—NH₂ membrane supported on flexible carbon cloth substrate was fabricated by a secondary growth method as summarised by FIG. 15 and elaborated in detail below. Post-synthetic defect healing was conducted on the as-prepared membranes to reduce the linker-missing defects of UiO-66(Zr)—NH₂. To verify defect-reduction took place, the healing process was performed on bulk UiO-66(Zr—NH₂) powder recovered from the mother liquor during membrane growth.

### Materials and Characterization

Zirconium (IV) chloride (ZrCl₄) and formic acid were purchased from Alfa Aesar. Benzoic acid and calcium chloride were purchased from Sinopharm Chemical Reagent Co, Ltd. 2-Aminoterephthalic acid (ATC) was purchased from Tee Hai Chem Pte Ltd. Carbon cloth was purchased from Hengqiu Technology, China. N,N-Dimethylformamide (DMF) was purchased from Avantor Performance Materials, Inc. Aluminum chloride (AlCl₃), methylene blue, and Nile red were purchased from TCI. Sodium chloride was purchased from VWR. 1,4-Dicarboxybenzene (BDC), oil red and anhydrous magnesium chloride were purchased from Sigma. All the chemicals were used without further purification.

Scanning electron microscope (SEM) images of the pure carbon cloth, seeded carbon cloth, and the prepared polycrystalline UiO-66(Zr)—NH₂ membranes were observed via a field-emission scanning electron microscope (FESEM, JSM-7610F, JEOL). The elements present in the prepared membranes were determined by EDS (Oxford Instruments, 80 mm² detector). Crystal phase was characterized by X-ray diffraction (XRD) on an X-ray powder diffractometer (Rigaku MiniFlex 600) at a scan rate of 1° min⁻¹. Water contact angles (WCAs) were measured using a contact angle meter (DSA30, US). Fourier transform infrared spectroscopy (FTIR) spectra were obtained with a Nicolet 6700 FTIR spectrometer.

### Carboxylation

Carbon cloth was initially carboxylated by placing the carbon cloth in a mixed solution containing nitric acid (65-68%) and hydrochloric acid (36-38%) (v/v=1/10) for one day, and then thoroughly rinsed with water and dried under vacuum for one day.

Carboxyl groups on the surface serve as anchoring sites for the growth of MOF layers. Therefore, carboxyl groups were introduced onto the surface of the carbon cloth by acid treatment. Fourier-transform infrared spectroscopy and water contact angle tests verified the successful introduction of carboxyl groups on the surface of the carbon cloth. It can be seen from the FTIR spectra that the carboxylated carbon cloth has peaks in the ranges of 2500-3300 and 1250-1428 cm⁻¹, representing the O—H bond in the carboxyl groups. In addition, a strong absorption peak at around 1700 cm⁻¹ can be found in the carboxylated carbon cloth, representing the expansion and contraction of C═O double bonds in the carboxyl groups.

### Seeding

The carboxylated carbon cloth substrate was seeded with UiO-66(Zr)—NH₂ crystals via in situ solvothermal method in a seeding solution with a molar ratio of 1 ZrCl₄/1 ATC/1 H₂O/500 DMF/100 benzoic acid. The seeding process was conducted in a Teflon-lined stainless steel autoclave at 120° C. for 1 day. After cooling to room temperature, the seeded carbon cloth was thoroughly washed with DMF and ethanol, and then dried at room temperature for further usage.

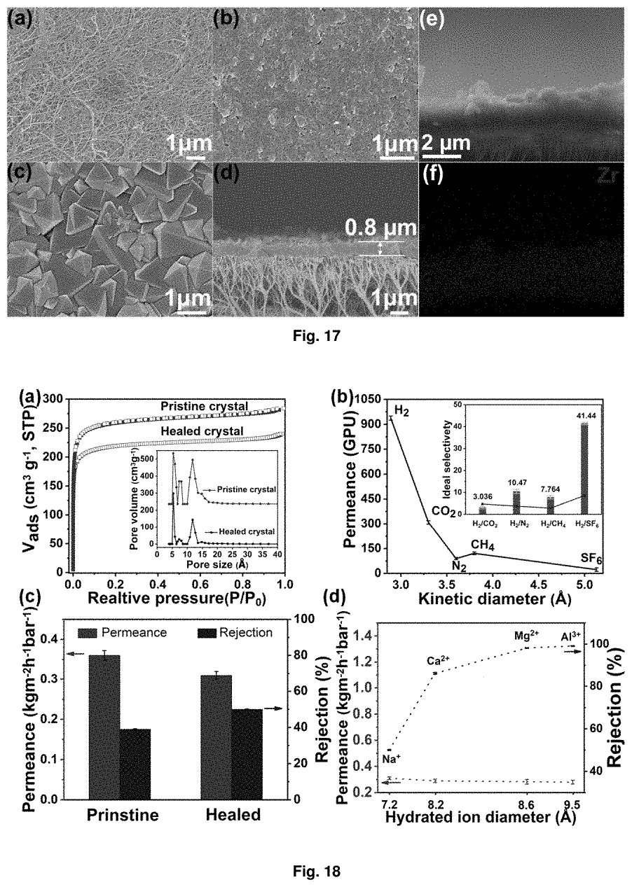

As shown by FIG. 16b and FIG. 17a-b, the substrate was coated with a crystalline and phase pure UiO-66(Zr)—NH₂ seed layer.

### Secondary Growth and Activation

A continuous and well-intergrown polycrystalline UiO-66(Zr)—NH₂ membrane can be obtained by a secondary growth method, in which the seeded substrate was treated in the growth solution with a molar ratio of 1 ZrCl₄/1 ATC/500 DMF/100 benzoic acid. The membrane growth was conducted at 120° C. for 3 days.

The prepared membranes were activated by soaking in fresh DMF for 12 h and repeating several times to ensure that the membrane surface does not have extra crystals deposited or residual reaction precursors. After that, the residual ligands and DMF were completely exchanged with hot ethanol before membrane performance tests.

UiO-66(Zr)—NH₂ powders were collected from the membrane growth solution after membrane preparation, and thoroughly washed with DMF and ethanol before further usage.

### Post-Synthetic Defect Healing

Post-synthetic defect healing was conducted by soaking the as-prepared polycrystalline MOF membranes or recovered MOF powders in healing solutions with a molar ratio of 2 ATC/500 DMF at 120° C. for 24 h.

### Characterization of Healed MOF Crystals

The healed crystals exhibit a reduced Brunauer-Emmett-Teller (BET) surface area (from 893 to 786 m² g⁻¹, FIG. 18a) and a reduction in average pore size (from 5.89 to 5.36 Å, FIG. 18a). N₂ sorption isotherms were measured using a surface area and pore size analyser (Micromeritics ASAP 2020).

The thermogravimetric analysis also indicates reduction in the proportion of missing linkers following the healing process (from 12.1% to 5.7%). The thermogravimetric analyses (TGA) were performed using a Shimadzu DTG-60AH instrument. Each TGA run was made in two different heating stages under a simultaneous feed of air (20 mL min⁻¹). In the first step, the samples were heated in the temperature range of 20-100° C. and kept for 30 min at a rate of ° C. min⁻¹; in the second step, the samples were continually heated to 950° C. at a rate of 5° C. min⁻¹.

Characterization of as-Prepared Membrane

As shown by the SEM images in FIG. 17c-d and XRD patterns in FIG. 16, well-intergrown polycrystalline UiO-66(Zr)—NH₂ membranes without any visible cracks or pinholes were fabricated. Notably, the size of the MOF crystals increased significantly under epitaxial growth during the growth step (FIG. 17c). Based on the cross-sectional scanning electron microscope (SEM) images, the average membrane thickness is around 0.8 μm, which is thinner than reported ceramic-supported Zr-MOF membranes (1-3.5 μm) [J. Am. Chem. Soc. 137 (2015) 6999-7002; ACS Appl. Mater. Interfaces 9 (2017) 37848-37855; and Adv. Funct. Mater. 27 (2017) 1604311].

Elemental mapping based on energy dispersive spectroscopy (EDS) was conducted to analyze the chemical composition of the membrane (FIG. 17e-f). Unlike Zr-MOF membranes grown on Al₂O₃ with distinct interface of Zr distribution between the selective layer and the substrate, strong Zr signal can also be observed from the carbon cloth substrate in the as-prepared membrane. It is thus believed that the UiO-66(Zr)—NH₂ crystals also grow inside the carbon cloth during the seeding step.

The water contact angle of the final UiO-66-NH₂ membrane was tested. The water contact angle of the membrane became 0° after 6 minutes, indicating that the final membrane is hydrophilic. In contrast, the water contact angle of the initial pristine carbon cloth could reach 130°, suggesting its high hydrophobicity.

The prepared UiO-66-NH₂ membrane displays negative surface zeta potentials of −22.1, −33.1, and −41.7 mV at pH=4, 7, and 10, respectively.

### Effect of Carboxylation of Carbon Substrate and Two-Step Process on Membrane Growth

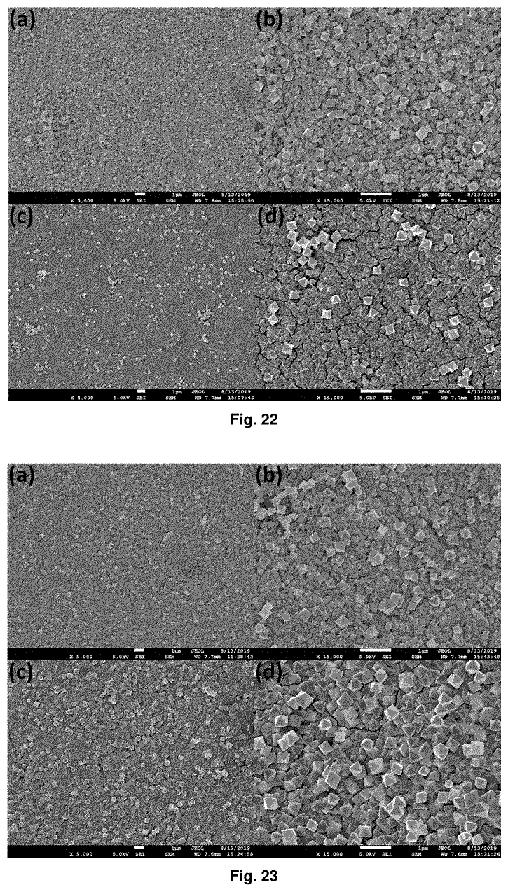

Polycrystalline UiO-66(Zr)—NH₂ membranes were grown in accordance with the procedures above except that a pristine carbon cloth was used with two-step growth (instead of a carboxylated carbon cloth). In another experiment, polycrystalline UiO-66(Zr)—NH₂ membranes were grown on a carboxylated cloth with direct growth. Both experiments did not yield high quality continuous membranes (FIGS. 21 and 22).

### Effect of Ligand on Membrane Growth

Polycrystalline UiO-66(Zr)—NH₂ membranes were grown in accordance with the procedures above except terephthalic acid (BDC) was selected as the ligand instead of aminoterephthalic acid (ATC). High quality continuous membranes were not obtained in spite of the use of a carboxylated carbon cloth with a two-step membrane growth procedure (FIG. 23).

The above results underline the importance of carboxylation in carbon cloth and two-step procedure in growing high quality polycrystalline Zr-MOF membranes on carbon cloth substrate, in which the selection of ligand also plays an important role.

### Example 5: Use of Healed Polycrystalline UiO-66-NH₂ Membrane for Gas Separation and Separation Under Aqueous Conditions

The membranes as prepared according to Example 4 was evaluated for gas separation and separation under aqueous conditions.

### Use of Healed Membrane for Single Gas Permeation

The integrity of the healed membranes was evaluated by single-gas permeation under a transmembrane pressure of 1.0 bar using H₂ (2.89 Å), CO₂ (3.3 Å), N₂ (3.64 Å), CH₄ (3.8 Å), and SF₆ (5.13 Å, FIG. 18b).

The single gas permeation performance of the membrane was tested at room temperature under a transmembrane pressure difference of 1.0 bar using a home-made Wicke-Kallenbach gas permeation apparatus reported in ACS Appl. Mater. Interfaces 9 (2017) 37848-37855. The volumetric gas flow rates were controlled by mass flow controllers (MFC, D07-26C, SevenStar, China). Argon with a volumetric flow rate of 50 mL min⁻¹ was used as the sweep gas. The molar concentrations of permeate side gas were analysed by a gas chromatograph (GC-2014, Shimadzu) with two TCD detectors. The permeation data were recorded at steady state when the composition concentrations of permeate side gas analysed by GC were constant. Every data point was tested at least three times to verify their reproducibility. The gas permeance (Pᵢ, GPU) and ideal selectivity (IS) of one gas over other gases were calculated by the following equations.

$${P}_{i} = \frac{{J}_{i}}{3.3928 \times 1 {0}^{10} Δ {P}_{i}} \qquad Eq . ( 3 )$$

where Jᵢ is the flux through membrane, mol m⁻² s⁻¹; ΔPᵢ is the transmembrane pressure difference of gases i, Pa.

$$IS = \frac{{P}_{i}}{{P}_{j}} \qquad Eq . ( 4 )$$

where Pᵢ and Pⱼ are the permeance of gas i and gas j, respectively.

As shown by the results in FIG. 18b, the gas permeance generally decreases with increasing size of the probe gas molecules. However, this trend is not strictly monotonic with regards to the kinetic diameter of the gas molecules. Because the aperture size of UiO-66-NH₂ (ca. 6.0 Å) is much larger than the kinetic diameters of H₂, CO₂, N₂, and CH₄, the separation performance can be attributed to a combination of size-selective and affinity-based factors.

The ideal permselectivities (IS) were calculated to be 3.036 for H₂/CO₂, 10.47 for H₂/N₂, 7.764 for H₂/CH₄, and 41.44 for H₂/SF₆.

### Liquid Separation Performance of Healed Membranes

Brackish water (0.2 wt. % NaCl, CaCl₂, MgCl₂, or AlCl₃) were used as feed solutions under a transmembrane pressure of 3.0 bar to evaluate the liquid separation performance and membrane stability.

Specifically, the tests were carried out in a dead-end system at room temperature under a transmembrane pressure of 3.0 bar. Before each test, the separation system was stabilized for 12 h to eliminate the adsorption effect. The filtrate was collected every 12 h, and each data point was tested for 3 times. The raw salt solution and filtrate concentrations were determined via a conductivity meter (D-82362 Wellheim). The ion rejections (R, %) were calculated as follows:

$$R = \frac{{C}_{f} - {C}_{p}}{{C}_{f}} \times 100 \% \qquad Eq . ( 5 )$$

where C_{f} and Cₚ are the ion concentrations in the feed and permeate solutions, respectively.

For the feed of NaCl aqueous solution, the liquid permeance of the healed membrane is around 0.31 kg m⁻² h⁻¹ bar⁻¹ with a rejection of around 50% (FIG. 18c). Notably, the membrane permeance is slightly higher than that of the reported polycrystalline UiO-66(Zr) membranes (0.14 and 0.286 kg m⁻² h⁻¹ bar⁻¹ for NaCl feed solution), which can be attributed to the reduced membrane thickness and increased hydrophilicity contributed by the amino groups of the ATC ligand.

The tests with other feeds containing multivalent cations indicate similar permeance but much higher rejection rates (86.2±0.4% for Ca²⁺, 98.2±0.1% for Mg²⁺, and 99.1±0.1% for Al³⁺, FIG. 18d), suggesting a strong molecular sieving effect.

### Example 6: Use of Polycrystalline UiO-66-NH₂ Membrane for Separations Involving Organic Solvents

Having confirmed the excellent membrane quality for the applications in aqueous conditions, the membranes prepared in accordance with Example 4 (that were subjected to post-synthetic defect healing) were tested for separations involving organic solvents or organic solvent nanofiltration. A series of filtration experiments were conducted on the prepared UiO-66(Zr)—NH₂ membrane using organic solutions containing dyes with various molecular weight, size, and charge, including methylene blue (MB, M_{w}=319.85 g mol⁻¹, 14.21×5.63 Å, cationic), oil red O (OR, M_{W}=408.495 g mol⁻¹, 19.39×10.34 Å, neutral), and Nile red (NR, M_{W}=318.37 g mol⁻¹, 14.12×6.51 Å, neutral).

### Experimental Set-Up

The dyes (methylene blue, MB; oil red 0, OR; and Nile red, NR) were dissolved in dichloromethane (CH₂Cl₂) or methanol with a concentration of 100 ppm to prepare the feeds.

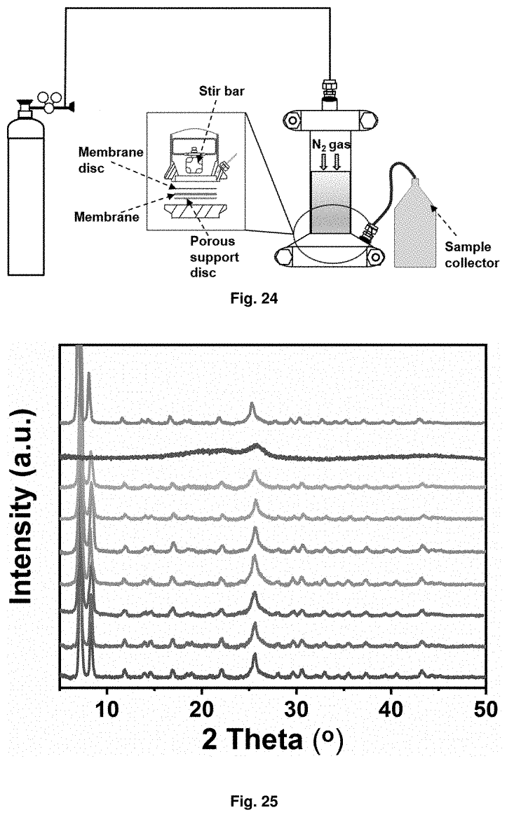

The OSN experiment was carried out in a dead-end system at room temperature under a transmembrane pressure of 6.0 bar (FIG. 24). Before each test, the separation system was stabilized for 12 h to eliminate the adsorption effect. In order to reduce the error of flux measurement, DMF (5 mL) was added into the feed solution, which could effectively reduce the evaporation loss of volatile organic solvents. The filtrate was collected every 12 h, and each data point was tested for 3 times. The dye concentrations in the feed and filtrate were determined via UV-Vis spectrometry.

In order to study the dye adsorption properties of the original and post-repaired UiO-66-NH₂ crystals, 20 mg of crystals were placed in 100 ppm Nile red methanol solution. After three days to reach adsorption equilibrium, the solution was tested by UV-Vis to determine the content of residual Nile red. Only about 1.0% of Nile red has been adsorbed by original and post-repaired UiO-66-NH₂ crystals, which is expected because the size of Nile red (14.12×6.51 Å) is larger than the aperture size of UiO-66-NH₂ (ca. 6.0 Å), and suggesting that the membrane separation performance should be due to diffusion and size exclusion mechanism.

The dye rejections (R, %) were calculated as Equation 5, with C, and C, representing the dye concentrations in the feed and permeate solutions, respectively. The permeance of organic solvent (P, kg m⁻² h⁻¹ bar⁻¹) was calculated as follows:

$$P = \frac{w}{A Δ t Δ P} \qquad Eq . ( 6 )$$

where w is the weight of collected organic solvent from the permeate side (kg); A is the effective membrane area (m²); Δt is the time (h); ΔP is the transmembrane pressure (bar).

Separation of Methylene Blue/Oil Red/Nile Red from Dichloromethane Solution

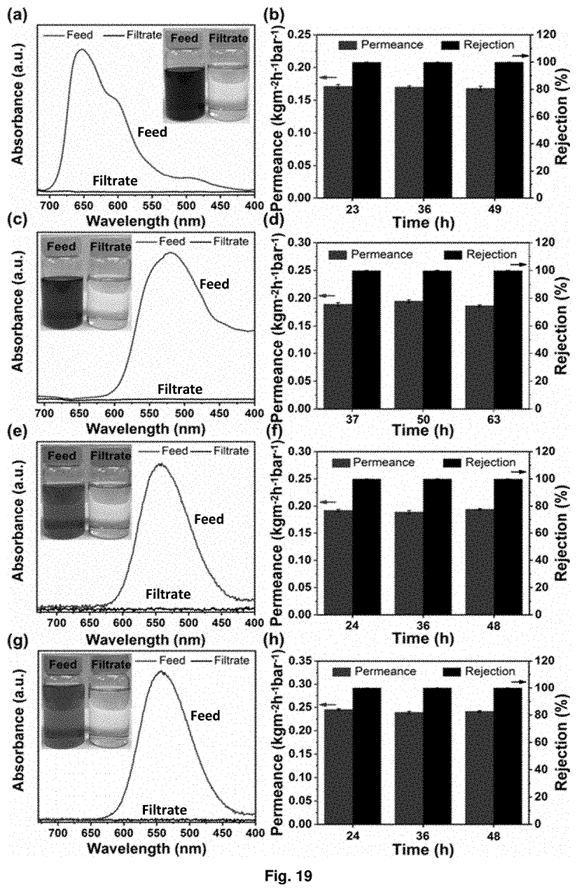

The dye solutions turned clear after passing through the UiO-66(Zr)—NH₂ membrane, indicating near-complete dye rejections. This was experimentally confirmed by the disappearance of the characteristic peaks corresponding to the dye molecules from the UV-Vis spectra of the filtrates. Using the Beer-Lambert law, the rejections of MB, OR, and NR in dichloromethane solutions were calculated to be 99.90, 99.95, and 99.85%, respectively (FIG. 19a-f). The dichloromethane permeance of MB, OR, and NR solutions was measured to be 0.17, 0.175, and 0.18 kg m⁻² h⁻¹ bar⁻¹, respectively.

Separation of Nile Red from Methanol Solution

The NR separation in methanol solution were also tested (FIG. 19g-h). Similar to the tests in dichloromethane solutions, near-complete rejection of NR was attained in the methanol solution. The methanol permeance of NR solution is 0.24 kg m⁻² h⁻¹ bar⁻¹.

Separation of Mixture of Oil Red and Methylene Blue from Methanol Solution

The membrane separation performance using a methanol solution containing both OR and MB (100 ppm each) were tested. The rejections of OR and MB were both higher than 99.9%, and the methanol permeance was 0.235 kg m⁻² h⁻¹ bar⁻¹, confirming good membrane separation performance.

### Membrane Stability Over Time and pH

The liquid flux and rejection remained almost unchanged over an operation period of >48 h during the filtration experiments in both aqueous and organic solvents (FIG. 19), indicating remarkable stability of the UiO-66(Zr)—NH₂ selective layer as well as the carbon cloth substrate.

X-ray diffraction (FIG. 25) and morphological characterization (not provided) further confirm the membrane stability under the relevant conditions. The XRD data indicate that the UiO-66(Zr)—NH₂ crystals are stable in NaCl aqueous solution (0.20 wt. %), aqueous solutions with a pH range of 1-11, and pure dichloromethane. It is noted that the membrane morphology starts to change in aqueous solutions with high pH values (around pH 13), indicating its instability under alkaline conditions similar to that of the bulk MOF crystals.

The original carbon cloth and the carbon cloth after solvothermal reaction were observed for three days by SEM (not provided). No morphological changes can be found between these two samples, indicating that the solvothermal reaction has no effect on the structure of the carbon cloth.

### 5-Day Continuous Separation Test

Dichloromethane solution containing 100 ppm OR was used as the separation liquid for a five-day continuous separation test, and no significant change in separation performance was observed (rejection rate>99.9%), indicating excellent solvent resistance. The high stability in water and organic solvents, as well as the excellent separation performance make the UiO-66(Zr)—NH₂ membrane an outstanding candidate for water treatment and OSN.

### Separation Performance of Membrane Variants

The separation performance of membranes variants prepared under the section “Effect of carboxylation of carbon substrate and two-step process on membrane growth” and “Effect of ligand on membrane growth” under Example 4 were evaluated and presented in Table 4. The tests were conducted using a similar set-up described in the current example at room temperature under a transmembrane pressure of 3 bar using methanol solutions containing 100 ppm of OR.

It is evident that the membrane variants exhibited poorer separation performance, which may be attributed to at least the absence of a continuous well-intergrown polycrystalline UiO-66(Zr)—NH₂ layer.

TABLE 4
Separation performance of UiO-66-NH₂ membranes
prepared under different conditions

| Preparation Conditions | Permeance (kg/m² h bar) | Rejection (%) |
|---|---|---|
| Non-Acidized Direct Growth | 30.8 | 33.2 |
| Non-Acidized Secondary | 5.97 | 96.3 |
| Growth | | |
| Acidized Direct Growth | 46.9 | 29.5 |
| UiO-66(BDC) Secondary | 12.6 | 89.6 |
| Growth | | |

Comparison of Membrane Performance with Reported Results

By way of background, various strategies have been developed to modify the established membrane configurations for OSN, such as integrally skinned asymmetric (ISA) membranes, thin film composite (TFC) membranes, and ceramic membranes. The performance of some reported OSN/SRNF membranes, along with the results of the as-prepared UiO-66(Zr)—NH₂ membranes (as discussed above) are summarized in Table 5. With few exceptions, the rejection of low molecular weight organics (M_{W} of ca. 300-400 g mol⁻¹) is difficult without substantially compromising the permeance in reported membranes.

TABLE 5
The separation performance of some state-of-the-art membranes

| Membrane | Membrane | | Permeance | | M_{w} | | |
|---|---|---|---|---|---|---|---|
| Type | Material | Solvent | [kg/(m² · h · bar)] | Marker | (g/mol) | Rejection | Ref |
| Polymeric | PA/PP | Methanol | 0.0948 | Safranin O | 351 | 45 | [17] |
| TFC | PA/PSf | Methanol | 1.58 | Bromothymol blue | 624 | >90 | [18] |
| Membranes | PS-b PEO/PAA | Methanol | 0.079 | Polyethylene glycols | 370 | 80 | [19] |
| Mixed Matrix | Polyimide | Acetone | 11.82 | Styrene oligomers | 1800 | 90 | [20] |
| Membranes | P84/HKUST-1 | | | | | | |
| | PDMS/(gold | Isopropanol | 0.0314 | Bromothymol blue | 624 | 97 | [21] |
| | nanoparticles)/PI | | | | | | |
| | Polyamide/MOFs | Methanol | 3.081 | Styrene oligomers | 236 | >90 | [22] |
| Ceramic | TiO₂/alumina | n-Hexane | 0.248 | Linoleic acid | 280.5 | 50 | [23] |
| Membranes | Grignard grafted | Acetone | 7.88 | Styrene oligomers | 580 | 85 | [24] |
| | TiO₂/alumina | | | | | | |
| | Inopor | Methanol | 0.316 | Victoria blue | 506 | 99 | [25] |
| | TiO₂/alumina | | | | | | |
| Polycrystalline | ZIF-8 | Ethanol | 1.1 | Rose bengal | 1018 | 83.9 | [26] |
| Membranes | | IPA | 0.3 | Rose bengal | 1018 | 93.5 | |
| | | Water | 5.6-37.5 | Rose bengal | 1018 | 98 | [27] |
| | UiO-66(Zr)—NH₂ | Dichloromethane | 0.17 | Methylene blue | 319 | 99.90 | This |
| | | | 0.18 | Oil red O | 408 | 99.95 | work |
| | | | 0.18 | Nile red | 318 | 99.85 | |
| | | Methanol | 0.24 | Nile red | 318 | 99.88 | |

17. P. B. Kosaraju, K. K. Sirkar, Interfacially polymerized thin film composite membranes on microporous polypropylene supports for solvent-resistant nanofiltration, J. Membr. Sci. 321 (2008) 155-161.
18. M. Peyravi, A. Rahimpour, M. Jahanshahi, Thin film composite membranes with modified polysulfone supports for organic solvent nanofiltration, J. Membr. Sci. 423 (2012) 225-237.
19. X. F. Li, W. Egger, I. F. J. Vankelecom, Ordered nanoporous membranes based on diblock copolymers with high chemical stability and tunable separation properties, J. Mater. Chem. 20 (2010) 4333-4339.
20. J. Campbell, G. Székely, A. G. Livingston, Fabrication of hybrid polymer/metal organic framework membranes: mixed matrix membranes versus in situ growth, J. Mater. Chem. A 2 (2014) 9260-9271.
21. Y. B. Li, T. Verbiest, I. Vankelecom, Improving the flux of PDMS membranes via localized heating through incorporation of gold nanoparticles, J. Membr. Sci. 428 (2013) 63-69.
22. S. Sorribas, P. Gorgojo, J. Coronas, A. G. Livingston, High flux thin film nanocomposite membranes based on metal-organic frameworks for organic solvent nanofiltration, J. Am. Chem. Soc. 135 (2013)15201-15208.
23. T. Tsuru, M. Narita, R. Shinagawa, T. Yoshioka, Nanoporous titania membranes for permeation and filtration of organic solutions, Desalination 233 (2008) 1-9.
24. S. R. Hosseinabadi, K. Wyns, R. Carleer, P. Adriaensens, A. Buekenhoudt, B. V. Bruggen, Organic solvent nanofiltration with Grignard functionalised ceramic nanofiltration membranes, J. Membr. Sci. 454 (2014) 496-504.
25. J. Geens, K. Boussu, C. Vandecasteele, B. V. Bruggen, Modelling of solute transport in non-aqueous nanofiltration, J. Membr. Sci. 281 (2006) 139-148.
26. Y. B. Li, L. H. Wee, A. Volodin, J. A. Martens, I. F. J. Vankelecom, Polymer supported ZIF-8 membranes prepared via an interfacial synthesis method, Chem. Commun. 51 (2015) 918-920.
27. Y. B. Li, L. H. Wee, A. Volodin, J. A. Martens, I. F. J. Vankelecom, Interfacial synthesis of ZIF-8 membranes with improved nanofiltration performance, J. Membr. Sci. 523 (2017) 561-566.

### Example 7: Bending Performance Test of Polycrystalline UiO-66-NH₂ Membrane

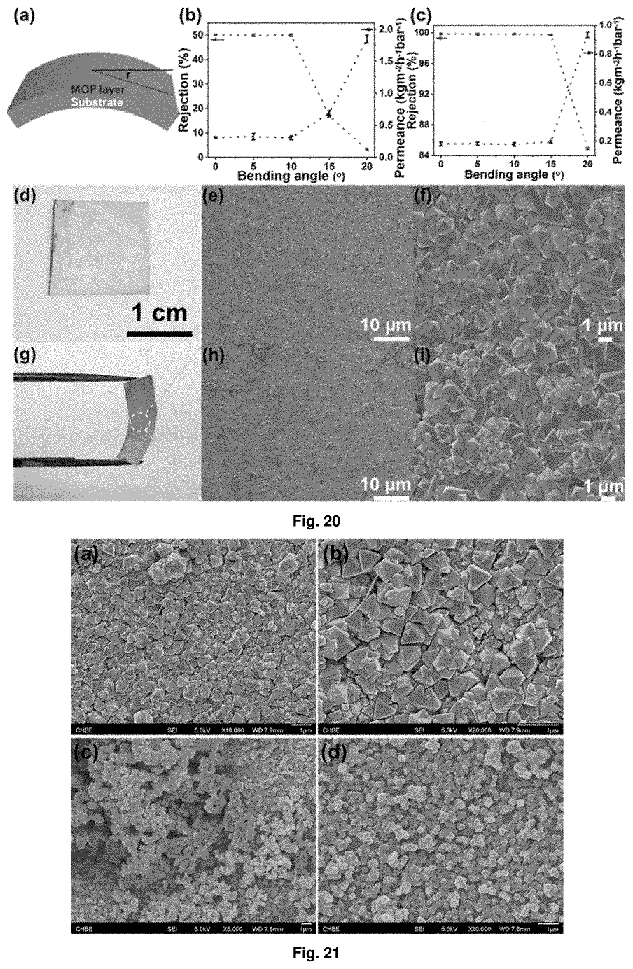

The bending tolerance of the polycrystalline UiO-66-NH₂ membrane was examined because this factor is important in designing membrane modules and increasing membrane packing density. The membrane separation performance was re-examined after varying degrees of bending, which is defined as the angular displacement of the ends of the membrane relative to the center (FIG. 20a).

### Bending Tolerance Test

In order to test the extent of bending the prepared UiO-66(Zr)—NH₂ membrane could withstand without damaging its separation performance, the bending tolerance test was performed by fixing the middle of the membrane as prepared according to Example 4 and then slowly bending the two sides to obtain a specific bending-angle. Each bending test was completed by bending the membrane three times under a specific bending-angle. After bending test, the separation performance of the membrane was evaluated using the experimental set-up described in Example 6, and each data point was tested for three times.

### Effect of Bending on Membrane Separation Performance

In the separation tests of brackish water (2 wt. % NaCl feed solution), the rejection and permeance remain almost unchanged when the bend angle is within 10°, indicating a good preservation of the membrane integrity within this bend angle range (FIG. 20b).

The bend tolerance of membrane using NR in dichloromethane as the feed solution was also tested, and the membrane performance can be preserved within a bend angle of 150 (FIG. 20c).

### Effect of Bending on Morphological Properties

The UiO-66(Zr)—NH₂ membrane after the 100 bending test was subject to morphological characterization, in which visible cracks could not be found from the large area SEM image (FIG. 20d-i).

### CONCLUSION

Overall, these results suggest that the polycrystalline UiO-66(Zr)—NH₂ membrane grown on carbon cloth substrate exhibits a certain flexibility (with bending-angle<10°) without affecting its separation performance, which should come from the flexible substrate and the framework dynamics of UiO-66-type MOFs. However, it should be noted that this level of flexibility is still not up to the degree required by spiral wound membranes. A short-term solution would be to apply these membranes in plate and frame configurations for OSN applications once the membrane sealing issues can be fully addressed.

1. A polycrystalline metal-organic framework membrane comprising:
a substrate material having a surface; and
a polycrystalline metal-organic framework attached to the surface of the substrate material, wherein the polycrystalline metal-organic framework is formed from:
a secondary building unit having the formula Ia or Ib:
M₆O₄(OH)₄ Ia,
where M is selected from Zr, Hf, and Ti; or
M′₆(OH)₈ Ib
where M′ is selected from Sm, Y, Dy, Er, Gd, and Ce; and
a ligand selected from fumaric acid, butynedioic acid, squaric acid, naphthalene-2,6-dicarboxylic acid, [2,2′-bipyridine]-5,5′-dicarboxylic acid, or a ligand having formula II: 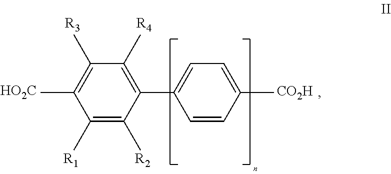
where:
R₁ is selected from H, halo, OR₅ₐ, SR_{5b}, C₁ to C₅ alkyl, NO₂, NR_{5c}R_{5d}, SO₃H, CF₃, or CO₂H;
R₂ is selected from H, halo, OR₆ₐ, SR_{6b}, C₁ to C₅ alkyl, NO₂, NR_{6c}R_{6d}, SO₃H, CF₃ or CO₂H;
R₃ is selected from H, halo, OR₇ₐ, SR_{7b}, C₁ to C₅ alkyl, NO₂, NR_{7c}R_{7d}, SO₃H, CF₃, or CO₂H;
R₄ is selected from H, halo, OR₈ₐ, SR_{8b}, C₁ to C₅ alkyl, NO₂, NR_{8c}R_{8d}, SO₃H, CF₃ or CO₂H;
R_{5a-5d}, R_{6a-6d}, R_{7a-7d}, R_{7a-7d} are each independently selected from H or C₁ to C₅ alkyl; or
R₁ and R₂ or R₃ and R₄ form, together with the carbon atoms to which they are attached to a C₆ aromatic ring; and n is 0, 1 or 2.

2. The membrane according to claim 1, wherein the substrate material is selected from one or more of a polymer, a ceramic, a carbon cloth, a metal, and a metal oxide.

3. The membrane according to claim 2, wherein, when the substrate material is alumina, then the secondary building unit has formula Ia, where M is Zr, Hf or Ti, or the secondary building unit has formula Ib.

4. The membrane according to claim 1, wherein the polycrystalline metal-organic framework is a UiO-66-type metal-organic framework.

5. The membrane according to claim 1, wherein:
R₁ is selected from H, halo, OR₅ₐ, SR_{5b}, C₁ to C₅ alkyl, NO₂, NR_{5c}R_{5d}, SO₃H, CF₃, or CO₂H;
R₂ is selected from H, OR₆ₐ, SR_{6b}, CF₃ or CO₂H;
R₃ is selected from H, halo, OR₇a, SR₇b, C₁ to C₅ alkyl, NR_{7c}R_{7d}, SO₃H, CF₃, or CO₂H; and
R₄ is selected from H, F, OR₈ₐ, SR_{8b}, CF₃ or CO₂H.

6. The membrane according to claim 1, wherein:
when two or more of R₁ to R₄ are not H, then the non-H substituents are identical to each other; and/or
n is 0 or 1.

7. The membrane according to claim 1, wherein the ligand is selected from fumaric acid, butynedioic acid, squaric acid, naphthalene-2,6-dicarboxylic acid, [2,2′-bipyridine]-5,5′-dicarboxylic acid, terephthalic acid, 2-fluoroterephthalic acid, 2-chloroterephthalic acid, 2-bromoterephthalic acid, 2-iodoterephthalic acid, 2-hydroxyterephthalic acid, 2-mercaptoterephthalic acid, 2-methylterephthalic acid, 2-nitroterephthalic acid, 2-aminoterephthalic acid, 2-sulfoterephthalic acid, 2-(trifluoromethyl)terephthalic acid, benzene-1,2,4-tricarboxylic acid, 2,3-dihydroxyterephthalic acid, 2,3-dimercaptoterephthalic acid, 2,5-difluoroterephthalic acid, 2,5-dichloroterephthalic acid, 2,5-dibromoterephthalic acid, 2,5-diiodoterephthalic acid, 2,5-terephthalic acid, 2,5-dihydroxyterephthalic acid, 2,5-dimercaptoterephthalic acid, 2,5-dimethylterephthalic acid, 2,5-diaminoterephthalic acid, 2,5-bis(trifluoromethyl)terephthalic acid, 2,5-diemthoxyterephthalic acid, benzene-1,2,4,5-tetracarboxylic acid, 2,5-disulfoterephthalic acid, 2,5-diethoxyterephthalic acid, 2,5-diisopropylterephthalic acid, 2,3,5,6-tetrafluoroterephthalic acid, 2,3,5,6-tetrahydroxyterephthalic acid, 2,3,5,6-tetramethylterephthalic acid, 2,3,5,6-tetrakis(trifluoromethyl)terephthalic acid, benzene-1,2,3,4,5,6-hexacarboxylic acid, [1,1′-biphenyl]-4,4′-dicarboxylic acid, [1,1′:4′,1″-terphenyl]-4,4″-dicarboxylic acid, and naphthalene-1,4-dicarboxylic acid.

8. The membrane according to claim 7, wherein the ligand is selected from or 2-aminoterephthalic acid or 2,5-dihydroxyterephthalic acid.

9. The membrane according to claim 1, wherein the substrate material is provided in the form of a mesh, a sheet or in the form of hollow fibers and other arrangements that are obtainable by the folding of a mesh, a sheet and hollow fibers.

10. The membrane according to claim 1, wherein the polycrystalline metal-organic framework attached to the surface of the substrate material has a thickness of from 20 nm to 20 μm.

11. A method of using a polycrystalline metal-organic framework membrane as described in claim 1 in a process of separating a fluid into a filtrate fluid and a retentate fluid, the process comprising the steps of:
(a) providing a fluid in need of separation to the polycrystalline metal-organic framework membrane;
(b) allowing or enabling a portion of the fluid to pass through the polycrystalline metal-organic framework membrane to provide a filtrate fluid and thereby providing a filtrate fluid; and
(c) collecting the filtrate fluid and retentate fluids.

12. The method according to claim 11, wherein the fluid to be separated is selected from: a mixture of gases; an aqueous solution comprising one or more inorganic materials; an aqueous solution comprising one or more organic materials; an aqueous solution comprising one or more inorganic materials and one or more organic materials; a mixture of organic liquids; a mixture of one or more organic liquids and water; a mixture of one or more organic liquids and one or more organic materials; a mixture of one or more organic liquids and one or more inorganic materials; a mixture of one or more organic liquids, one or more organic materials and one or more inorganic materials; a mixture of water, one or more organic liquids and one or more organic materials; a mixture of water, one or more organic liquids and one or more inorganic materials; and a mixture of water, one or more organic liquids, one or more organic materials and one or more inorganic materials.

13. A method of forming a polycrystalline metal-organic framework membrane as described in claim 1, the method comprising the steps of:
providing a seeded substrate having a surface seeded with seed crystals of the metal-organic framework, said seed crystals formed from a secondary building unit having the formula Ia or Ib and the ligand selected from fumaric acid, butynedioic acid, squaric acid, naphthalene-2,6-dicarboxylic acid, [2,2′-bipyridine]-5,5′-dicarboxylic acid, or the ligand having formula II; and
subjecting the seeded substrate to a first mother liquor comprising a solvent, a metal salt precursor and a ligand selected from fumaric acid, butynedioic acid, squaric acid, naphthalene-2,6-dicarboxylic acid, [2,2′-bipyridine]-5,5′-dicarboxylic acid, or a ligand having formula II as described above, for a first period of time under conditions sufficient to form a metal-organic framework membrane, wherein the metal salt precursor and the ligand are selected to form the same metal-organic framework as in the seed crystals.

14. The method of claim 13, wherein the seeded substrate is formed by immersing a substrate having a surface in a second mother liquor that comprises a solvent, a metal salt precursor and the ligand selected from fumaric acid, butynedioic acid, squaric acid, naphthalene-2,6-dicarboxylic acid, [2,2′-bipyridine]-5,5′-dicarboxylic acid, or the ligand having formula II for a second period of time to provide a seeded substrate having a surface seeded with seed crystals of the metal organic framework, said seed crystals formed from a secondary building unit having the formula Ia or Ib and the ligand selected from fumaric acid, butynedioic acid, squaric acid, naphthalene-2,6-dicarboxylic acid, [2,2′-bipyridine]-5,5′-dicarboxylic acid, or the ligand having formula II.

15. A method of post-synthetic defect healing comprising the steps of:
(a) providing a polycrystalline metal-organic framework membrane in need of post-synthetic healing formed by the process of claim 13;
(b) subjecting the polycrystalline metal-organic framework membrane in need of post-synthetic healing to a solution comprising a solvent the ligand selected from fumaric acid, butynedioic acid, squaric acid, naphthalene-2,6-dicarboxylic acid, [2,2′-bipyridine]-5,5′-dicarboxylic acid, or the ligand having formula II, for a period of time under conditions sufficient to achieve the post-synthetic healing, wherein the ligand are selected to form the same metal-organic framework as in the polycrystalline metal-organic framework membrane.

16. The method according to claim 15, wherein the polycrystalline metal-organic framework membrane is formed from a secondary building unit of formula Ia, where M is Zr and the ligand is 2-aminoterephthalic acid.

17. A method of in situ healing, wherein the method comprises the steps of:
(a) providing a damaged polycrystalline metal-organic framework membrane, where the polycrystalline metal-organic framework membrane is as described in claim 1; and
(b) subjecting the damaged polycrystalline metal-organic framework membrane to a solution comprising a reaction solution comprising a solvent, a metal salt precursor, the ligand selected from fumaric acid, butynedioic acid, squaric acid, naphthalene-2,6-dicarboxylic acid, [2,2′-bipyridine]-5,5′-dicarboxylic acid, or the ligand having formula II, and reactive rare earth-containing secondary building units for a period of time under conditions sufficient to heal the damaged polycrystalline metal-organic framework membrane, wherein the metal salt precursor, the ligand and the rare earth-containing secondary building units are selected to form the same metal-organic framework as in the damaged polycrystalline metal-organic framework membrane.

18. The method according to claim 15, wherein the damaged polycrystalline metal-organic framework membrane is formed from a secondary building unit that has formula Ib, where M′ is Sm and the ligand is 2,5-dihydroxyterephthalic acid.
